(19) United States
(12) Patent Application Publication
(10) Pub. No.: US 2026/0207343 A1
(43) Pub. Date: Jul. 23, 2026

(54) IMPLANTS, INSTRUMENTS, AND METHODS OF USE FOR ANKLE ARTHROPLASTY PROCEDURES

(71) Applicant: Paragon 28, Inc., Englewood, CO (US)

(72) Inventors: Darby MACLEISH, Denver, CO (US); Dylan PAPROSKI, Littleton, CO (US); Caitlin REUTER, Littleton, CO (US); Daniel J. LEE, Denver, CO (US); Zachary Ryan WOODS, Centennial, CO (US)

(73) Assignee: Paragon 28, Inc., Englewood, CO (US)

(21) Appl. No.: 19/572,472

(22) Filed: Mar. 19, 2026

Related U.S. Application Data
(63) Continuation of application No. PCT/US2024/047477, filed on Sep. 19, 2024.
(60) Provisional application No. 63/583,828, filed on Sep. 19, 2023.
(60) Provisional application No. 63/605,184, filed on Dec. 1, 2023.

(51) Int. Cl. A61F 2/42; A61F 2/46

(52) U.S. Cl. CPC A61F 2/4202; A61F 2/4606; A61F 2002/4205; A61F 2002/4687

(57) ABSTRACT

An ankle arthroplasty implant having a body. The body includes a top surface opposite a bottom surface, a lateral surface opposite a medial surface, and a front end opposite a rear end. The implant also includes an extension extending upward from a central portion of the top surface of the body. The extension includes a rounded top portion separated from the top surface of the implant by a shaft. Also disclosed is an ankle arthroplasty kit that includes a plurality of tibial implants, plurality of poly bearing inserts for insertion into the tibial implant. The kit also includes various instrumentation, including a tibial alignment guide, a drill guide and a poly bearing insert inserter.

### CROSS RELATED APPLICATIONS

This is a bypass continuation application which claims priority of International PCT Application No. PCT/US2024/047477 filed Sep. 19, 2024, and entitled “Implants, Instruments, and Methods of Use For Ankle Arthroplasty Procedures,” which claims priority benefit of U.S. Provisional Application No. 63/583,828 filed on Sep. 19, 2023, and entitled “Implants, Instruments, and Methods of Use For Ankle Arthroplasty Procedures,” and U.S. Provisional Application No. 63/605,184 filed on Dec. 1, 2023, and entitled “Implants, Instruments, and Methods of Use For Ankle Arthroplasty Procedures,” the disclosures of each of these applications are all hereby incorporated herein by reference in their entirety.

### FIELD OF INVENTION

The present disclosure relates to surgical implants, instruments, guides, and methods of use to be implemented in surgical procedures. The present disclosure relates to podiatric and orthopedic surgical implants, instruments, guides, and methodology to be implemented in various procedures of the foot and/or ankle, for example procedures to address the ankle joint. More specifically, but not exclusively, the present disclosure relates to surgical implants, instruments, guides, and methodology to be implemented in arthroplasty procedures of the ankle joint.

### BACKGROUND OF THE INVENTION

Many currently available surgical implants, instruments and guides, as well as methodology, do not completely address the needs of patients. Additionally, many currently available surgical implants, instruments, guides, and methodology fail to account for properties of joint anatomy and accordingly can decrease favorability of the outcome for the patient.

### SUMMARY OF THE INVENTION

The present disclosure is directed toward implants, instrumentation, guides, and methodology to be implemented in procedures of the foot and ankle, including the ankle joint.

A first aspect of the present disclosure is an ankle arthroplasty implant, which includes a body. The body includes a top surface opposite a bottom surface, a lateral surface opposite a medial surface, and a front end opposite a rear end. The implant also includes an extension extending upward from a central portion of the top surface of the body, wherein the extension includes a rounded top portion separated from the top surface of the implant by a shaft.

A second aspect of the present disclosure is an ankle arthroplasty implant having a body which may include an at least partially porous lattice structure extending through at least a portion of the body. The body may have a top surface opposite a bottom surface, a lateral surface opposite a medial surface and a front end opposite a rear end. The body may also include an extension extending upward from a central portion of the top surface of the body. The extension may have a shaft portion integral with the body and be comprised of a substantially cylindrical geometry. The extension may also include a top portion positioned at and integral with a terminal end of the shaft portion. The top portion has a rounded geometry along at least a portion of an outer surface. The extension also may include a base portion that extends upward from and is integral with the body of the implant. The base portion extends around at least a portion of an outer surface of the shaft portion, with the base portion having at least partially porous lattice structure through at least a portion of the base portion.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a top, front perspective view of an exemplary implant of an ankle arthroplasty system, in accordance with the present disclosure;

FIG. 2 is a top, rear perspective of the exemplary implant of FIG. 1 of an ankle arthroplasty system, in accordance with the present disclosure;

FIG. 3 is front view of the exemplary implant of FIG. 1 of an ankle arthroplasty system, in accordance with the present disclosure;

FIG. 4 is a rear view of the exemplary implant of FIG. 1 of an ankle arthroplasty system, in accordance with the present disclosure;

FIG. 5 is a top view of the exemplary implant of FIG. 1 of an ankle arthroplasty system, in accordance with the present disclosure;

FIG. 6 is a bottom view of the exemplary implant of FIG. 1 of an ankle arthroplasty system, in accordance with the present disclosure;

FIG. 7 is a side view of the exemplary implant of FIG. 1 of an ankle arthroplasty system, in accordance with the present disclosure;

FIG. 8 is an alternate side view of the exemplary implant of FIG. 1 of an ankle arthroplasty system, in accordance with the present disclosure;

FIG. 9 is a top, front perspective view of an exemplary implant of an ankle arthroplasty system, in accordance with the present disclosure;

FIG. 10 is a top, rear perspective of the exemplary implant of FIG. 9 of an ankle arthroplasty system, in accordance with the present disclosure;

FIG. 11 is front view of the exemplary implant of FIG. 9 of an ankle arthroplasty system, in accordance with the present disclosure;

FIG. 12 is a rear view of the exemplary implant of FIG. 9 of an ankle arthroplasty system, in accordance with the present disclosure;

FIG. 13 is a top view of the exemplary implant of FIG. 9 of an ankle arthroplasty system, in accordance with the present disclosure;

FIG. 14 is a bottom view of the exemplary implant of FIG. 9 of an ankle arthroplasty system, in accordance with the present disclosure;

FIG. 15 is a side view of the exemplary implant of FIG. 9 of an ankle arthroplasty system, in accordance with the present disclosure;

FIG. 16 is an alternate side view of the exemplary implant of FIG. 9 of an ankle arthroplasty system, in accordance with the present disclosure;

FIG. 17 is a top, front perspective view of an exemplary implant of an ankle arthroplasty system, in accordance with the present disclosure;

FIG. 18 is a top, rear perspective of the exemplary implant of FIG. 17 of an ankle arthroplasty system, in accordance with the present disclosure;

FIG. 19 is front view of the exemplary implant of FIG. 17 of an ankle arthroplasty system, in accordance with the present disclosure;

FIG. 20 is a rear view of the exemplary implant of FIG. 17 of an ankle arthroplasty system, in accordance with the present disclosure;

FIG. 21 is a top view of the exemplary implant of FIG. 17 of an ankle arthroplasty system, in accordance with the present disclosure;

FIG. 22 is a bottom view of the exemplary implant of FIG. 17 of an ankle arthroplasty system, in accordance with the present disclosure;

FIG. 23 is a side view of the exemplary implant of FIG. 17 of an ankle arthroplasty system, in accordance with the present disclosure;

FIG. 24 is an alternate side view of the exemplary implant of FIG. 17 of an ankle arthroplasty system, in accordance with the present disclosure;

FIG. 25 is a top, front perspective view of an exemplary implant of an ankle arthroplasty system, in accordance with the present disclosure;

FIG. 26 is a top, rear perspective of the exemplary implant of FIG. 25 of an ankle arthroplasty system, in accordance with the present disclosure;

FIG. 27 is front view of the exemplary implant of FIG. 25 of an ankle arthroplasty system, in accordance with the present disclosure;

FIG. 28 is a rear view of the exemplary implant of FIG. 25 of an ankle arthroplasty system, in accordance with the present disclosure;

FIG. 29 is a top view of the exemplary implant of FIG. 25 of an ankle arthroplasty system, in accordance with the present disclosure;

FIG. 30 is a bottom view of the exemplary implant of FIG. 25 of an ankle arthroplasty system, in accordance with the present disclosure;

FIG. 31 is a side view of the exemplary implant of FIG. 25 of an ankle arthroplasty system, in accordance with the present disclosure;

FIG. 32 is an alternate side view of the exemplary implant of FIG. 25 of an ankle arthroplasty system, in accordance with the present disclosure;

FIG. 33 is a top, front perspective view of an exemplary implant of an ankle arthroplasty system, in accordance with the present disclosure;

FIG. 34 is a top, rear perspective of the exemplary implant of FIG. 33 of an ankle arthroplasty system, in accordance with the present disclosure;

FIG. 35 is front view of the exemplary implant of FIG. 33 of an ankle arthroplasty system, in accordance with the present disclosure;

FIG. 36 is a rear view of the exemplary implant of FIG. 33 of an ankle arthroplasty system, in accordance with the present disclosure;

FIG. 37 is a top view of the exemplary implant of FIG. 33 of an ankle arthroplasty system, in accordance with the present disclosure;

FIG. 38 is a bottom view of the exemplary implant of FIG. 33 of an ankle arthroplasty system, in accordance with the present disclosure;

FIG. 39 is a side view of the exemplary implant of FIG. 33 of an ankle arthroplasty system, in accordance with the present disclosure;

FIG. 40 is an alternate side view of the exemplary implant of FIG. 33 of an ankle arthroplasty system, in accordance with the present disclosure;

FIG. 41 is a top, front perspective view of an exemplary implant of an ankle arthroplasty system, in accordance with the present disclosure;

FIG. 42 is a top, rear perspective of the exemplary implant of FIG. 41 of an ankle arthroplasty system, in accordance with the present disclosure;

FIG. 43 is front view of the exemplary implant of FIG. 41 of an ankle arthroplasty system, in accordance with the present disclosure;

FIG. 44 is a rear view of the exemplary implant of FIG. 41 of an ankle arthroplasty system, in accordance with the present disclosure;

FIG. 45 is a top view of the exemplary implant of FIG. 41 of an ankle arthroplasty system, in accordance with the present disclosure;

FIG. 46 is a bottom view of the exemplary implant of FIG. 41 of an ankle arthroplasty system, in accordance with the present disclosure;

FIG. 47 is a side view of the exemplary implant of FIG. 41 of an ankle arthroplasty system, in accordance with the present disclosure;

FIG. 48 is an alternate side view of the exemplary implant of FIG. 41 of an ankle arthroplasty system, in accordance with the present disclosure;

FIG. 49 is a top, front perspective view of an exemplary implant of an ankle arthroplasty system, in accordance with the present disclosure;

FIG. 50 is a top, rear perspective of the exemplary implant of FIG. 49 of an ankle arthroplasty system, in accordance with the present disclosure;

FIG. 51 is front view of the exemplary implant of FIG. 49 of an ankle arthroplasty system, in accordance with the present disclosure;

FIG. 52 is a rear view of the exemplary implant of FIG. 49 of an ankle arthroplasty system, in accordance with the present disclosure;

FIG. 53 is a top view of the exemplary implant of FIG. 49 of an ankle arthroplasty system, in accordance with the present disclosure;

FIG. 54 is a bottom view of the exemplary implant of FIG. 49 of an ankle arthroplasty system, in accordance with the present disclosure;

FIG. 55 is a side view of the exemplary implant of FIG. 49 of an ankle arthroplasty system, in accordance with the present disclosure;

FIG. 56 is an alternate side view of the exemplary implant of FIG. 49 of an ankle arthroplasty system, in accordance with the present disclosure;

FIG. 57 is a top, front perspective view of an exemplary implant of an ankle arthroplasty system, in accordance with the present disclosure;

FIG. 58 is a top, rear perspective of the exemplary implant of FIG. 58 of an ankle arthroplasty system, in accordance with the present disclosure;

FIG. 59 is front view of the exemplary implant of FIG. 58 of an ankle arthroplasty system, in accordance with the present disclosure;

FIG. 60 is a rear view of the exemplary implant of FIG. 58 of an ankle arthroplasty system, in accordance with the present disclosure;

FIG. 61 is a top view of the exemplary implant of FIG. 58 of an ankle arthroplasty system, in accordance with the present disclosure;

FIG. 62 is a bottom view of the exemplary implant of FIG. 58 of an ankle arthroplasty system, in accordance with the present disclosure;

FIG. 63 is a side view of the exemplary implant of FIG. 58 of an ankle arthroplasty system, in accordance with the present disclosure;

FIG. 64 is an alternate side view of the exemplary implant of FIG. 58 of an ankle arthroplasty system, in accordance with the present disclosure;

FIG. 65 is a front perspective of an exemplary instrument, in accordance with the present disclosure;

FIG. 66 is rear perspective view of the exemplary instrument of FIG. 65, in accordance with the present disclosure;

FIG. 67 is a side view of the exemplary instrument of FIG. 65, in accordance with the present disclosure;

FIG. 68 is an alternate side view of the exemplary instrument of FIG. 65, in accordance with the present disclosure;

FIG. 69 is a front view of the exemplary instrument of FIG. 65, in accordance with the present disclosure;

FIG. 70 is a side view of a portion of the exemplary instrument of FIG. 65, in accordance with the present disclosure;

FIG. 71 is an alternate side view of a portion of the exemplary instrument of FIG. 65, in accordance with the present disclosure;

FIG. 72 is a front perspective of an exemplary instrument, in accordance with the present disclosure;

FIG. 73 is rear perspective view of the exemplary instrument of FIG. 72, in accordance with the present disclosure;

FIG. 74 is a side view of the exemplary instrument of FIG. 72, in accordance with the present disclosure;

FIG. 75 is a top, front perspective view of an exemplary instrument of an ankle arthroplasty system, in accordance with the present disclosure;

FIG. 76 is a top, rear perspective of the exemplary instrument of FIG. 75 of an ankle arthroplasty system, in accordance with the present disclosure;

FIG. 77 is front view of the exemplary instrument of FIG. 75 of an ankle arthroplasty system, in accordance with the present disclosure;

FIG. 78 is a rear view of the exemplary instrument of FIG. 75 of an ankle arthroplasty system, in accordance with the present disclosure;

FIG. 79 is a top view of the exemplary instrument of FIG. 75 of an ankle arthroplasty system, in accordance with the present disclosure;

FIG. 80 is a bottom view of the exemplary instrument of FIG. 75 of an ankle arthroplasty system, in accordance with the present disclosure;

FIG. 81 is a side view of the exemplary instrument of FIG. 75 of an ankle arthroplasty system, in accordance with the present disclosure;

FIG. 82 is an alternate side view of the exemplary instrument of FIG. 75 of an ankle arthroplasty system, in accordance with the present disclosure;

FIG. 83 is a top, front perspective view of an exemplary instrument of an ankle arthroplasty system, in accordance with the present disclosure;

FIG. 84 is a top, rear perspective of the exemplary instrument of FIG. 84 of an ankle arthroplasty system, in accordance with the present disclosure;

FIG. 85 is front view of the exemplary instrument of FIG. 84 of an ankle arthroplasty system, in accordance with the present disclosure;

FIG. 86 is a rear view of the exemplary instrument of FIG. 84 of an ankle arthroplasty system, in accordance with the present disclosure;

FIG. 87 is a top view of the exemplary instrument of FIG. 84 of an ankle arthroplasty system, in accordance with the present disclosure;

FIG. 88 is a bottom view of the exemplary instrument of FIG. 84 of an ankle arthroplasty system, in accordance with the present disclosure;

FIG. 89 is a side view of the exemplary instrument of FIG. 84 of an ankle arthroplasty system, in accordance with the present disclosure;

FIG. 90 is an alternate side view of the exemplary instrument of FIG. 84 of an ankle arthroplasty system, in accordance with the present disclosure;

FIG. 91 is a top, front perspective view of an exemplary instrument of an ankle arthroplasty system, in accordance with the present disclosure;

FIG. 92 is a top, rear perspective of the exemplary instrument of FIG. 91 of an ankle arthroplasty system, in accordance with the present disclosure;

FIG. 93 is front view of the exemplary instrument of FIG. 91 of an ankle arthroplasty system, in accordance with the present disclosure;

FIG. 94 is a rear view of the exemplary instrument of FIG. 91 of an ankle arthroplasty system, in accordance with the present disclosure;

FIG. 95 is a top view of the exemplary instrument of FIG. 91 of an ankle arthroplasty system, in accordance with the present disclosure;

FIG. 96 is a bottom view of the exemplary instrument of FIG. 91 of an ankle arthroplasty system, in accordance with the present disclosure;

FIG. 97 is a side view of the exemplary instrument of FIG. 91 of an ankle arthroplasty system, in accordance with the present disclosure;

FIG. 98 is an alternate side view of the exemplary instrument of FIG. 91 of an ankle arthroplasty system, in accordance with the present disclosure;

FIG. 99 is an elevated side perspective view of an exemplary instrument of an ankle arthroplasty system, in accordance with the present disclosure;

FIG. 100 is an alternate elevated side perspective view of the exemplary instrument of FIG. 99 of an ankle arthroplasty system, in accordance with the present disclosure;

FIG. 101 is a top view of the exemplary instrument of FIG. 99 of an ankle arthroplasty system, in accordance with the present disclosure;

FIG. 102 is a bottom view of the exemplary instrument of FIG. 99 of an ankle arthroplasty system, in accordance with the present disclosure;

FIG. 103 is a front view of the exemplary instrument of FIG. 99 of an ankle arthroplasty system, in accordance with the present disclosure;

FIG. 104 is a rear view of the exemplary instrument of FIG. 99 of an ankle arthroplasty system, in accordance with the present disclosure;

FIG. 105 is a side perspective view of the exemplary instrument of FIG. 99 of an ankle arthroplasty system, in accordance with the present disclosure;

FIG. 106 is an elevated front perspective view of an exemplary instrument of an ankle arthroplasty system, in accordance with the present disclosure;

FIG. 107 is an elevated rear perspective view of the exemplary instrument of FIG. 106 of an ankle arthroplasty system, in accordance with the present disclosure;

FIG. 108 is a front view of the exemplary instrument of FIG. 106 of an ankle arthroplasty system, in accordance with the present disclosure;

FIG. 109 is a rear view of the exemplary instrument of FIG. 106 of an ankle arthroplasty system, in accordance with the present disclosure;

FIG. 110 is a top view of the exemplary instrument of FIG. 106 of an ankle arthroplasty system, in accordance with the present disclosure;

FIG. 111 is a bottom view of the exemplary instrument of FIG. 106 of an ankle arthroplasty system, in accordance with the present disclosure;

FIG. 112 is a side view of the exemplary instrument of FIG. 106 of an ankle arthroplasty system, in accordance with the present disclosure;

FIG. 113 is an alternate side view of the exemplary instrument of FIG. 106 of an ankle arthroplasty system, in accordance with the present disclosure;

FIG. 114 is an elevated front perspective view of an exemplary instrument engaged with components of an ankle arthroplasty system, in accordance with the present disclosure;

FIG. 115 is an elevated front perspective view of the exemplary instrument of FIG. 114 of an ankle arthroplasty system, in accordance with the present disclosure;

FIG. 116 is a rear elevated perspective view of the exemplary instrument of FIG. 114 of an ankle arthroplasty system, in accordance with the present disclosure;

FIG. 117 is a top view of the exemplary instrument of FIG. 114 of an ankle arthroplasty system, in accordance with the present disclosure;

FIG. 118 is a bottom view of the exemplary instrument of FIG. 114 of an ankle arthroplasty system, in accordance with the present disclosure;

FIG. 119 is a front view of the exemplary instrument of FIG. 114 of an ankle arthroplasty system, in accordance with the present disclosure;

FIG. 120 is a rear view of the exemplary instrument of FIG. 114 of an ankle arthroplasty system, in accordance with the present disclosure;

FIG. 121 is a side view of the exemplary instrument of FIG. 114 of an ankle arthroplasty system, in accordance with the present disclosure; and

FIG. 122 is an alternate side view of the exemplary instrument of FIG. 114 of an ankle arthroplasty system, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, systems, assemblies and related methods disclosed in disclosed U.S. Pat. No. 10,117,749, issued on Nov. 6, 2018 and entitled Subtalar Joint Implant; European Patent No. 3756626 issued on Dec. 30, 2020 and entitled Subtalar Joint Implant, European Patent Application No. 15770960.1A filed on Jul. 15, 2020 and entitled Subtalar Joint Implant; U.S. Provisional Patent Application No. 63/155,100 filed on Mar. 1, 2021 and entitled Methods for Performing Arthroplasty of the Subtalar Joint; U.S. Provisional Patent Application No. 63/167,965 filed on Mar. 30, 2021 and entitled Orthopedic Implants and Methods; International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615 , filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; which are hereby incorporated herein by reference in their entireties. Similarly, the instruments, implants, systems, assemblies, and related methods for maintaining, correcting, and/or resurfacing joint surfaces of the present disclosure may include one or more instrument (e.g., one or more insertion and/or implantation instruments) disclosed in International PCT Application No. PCT/US2019/29009, filed on Apr. 24, 2019, and entitled Implants and Methods of Use and Assembly; International PCT Application No. PCT/US2019/64741, filed on Dec. 12, 2019, and entitled Implant System and Methods of Use; International PCT Application No. PCT/US2019/66336, filed on Dec. 13, 2019, and entitled Patient Specific Instrumentation and Methods of Use; and/or International PCT Application No. PCT/US2019/66408, filed on Dec. 13, 2019, and entitled Joint Replacement Alignment Guides, System, and Methods of Use and Assembly; and/or International PCT Application No. PCT/US2019/66149 filed on Dec. 13, 2019, and entitled Alignment Instruments and Methods for Use in Total Ankle Replacement; and/or International PCT Application No. PCT/US2019/66393, filed on Dec. 13, 2019, entitled Joint Replacement Alignment Guides, Systems, and Methods of Use and Assembly; and/or U.S. Provisional Patent Application No. 62/898,615 , filed on Sep. 11, 2019, entitled Resection Guides, Sweeping Reamers, and Methods for Use in Total Ankle Replacement; and/or International PCT Application No. PCT/US2019/66398, files on Dec. 13, 2019, entitled Distractors Having Attachable Paddles, Impaction Devices, and Methods for Use in Total Ankle Replacement; and/or International PCT Application No. PCT/US2019/65025, filed on Dec. 6, 2019, entitled Trial Insert Assembly; and/or U.S. Provisional Patent Application No. 62/899,460 filed Sep. 12, 2019, entitled Total Ankle Replacement Surgical Method; and/or International PCT Application No. PCT/US2019/66404 filed on Dec. 13, 2019, entitled Instruments, Guides, and Related Methods for Total Ankle Replacement; and/or U.S. patent application Ser. No. 16/672,505 filed Nov. 3, 2019 and titled Talus Formational And Implantation Method; and/or International PCT Application No. PCT/US2021/046920 and titled Whole Talus Implant and Method; and/or International PCT Application No. PCT/US2021/047117 and titled Implant for Focal Talus Defects and Method; and/or U.S. Provisional Patent Application No. 63/377,639 and titled Implant, Instrumentation, and Surgical Methods for Ankle Arthroplasty; which are hereby incorporated herein by reference in their entireties.

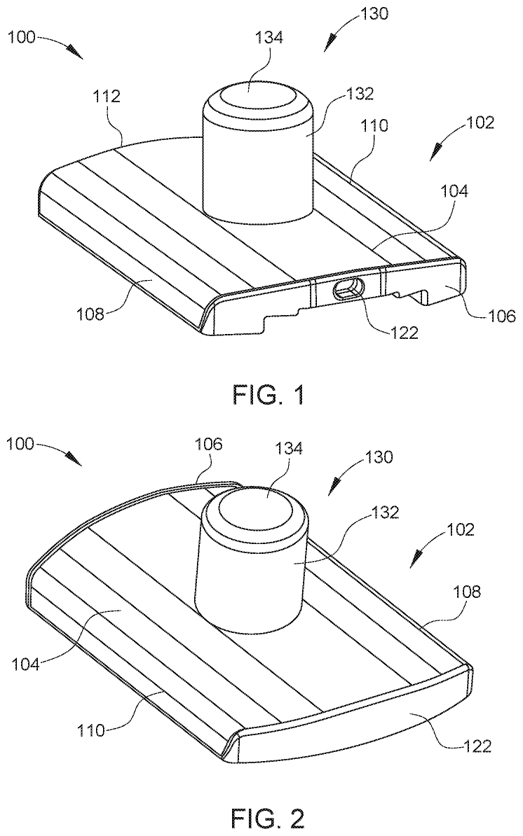

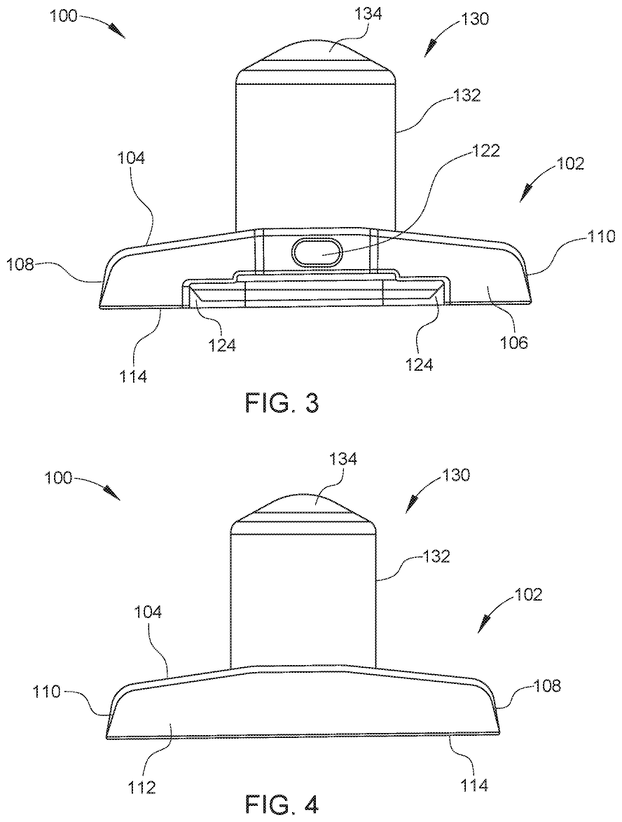

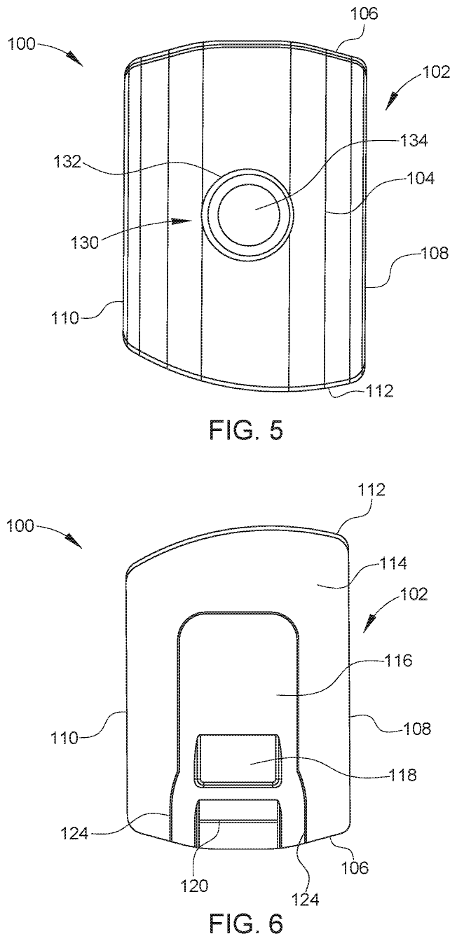

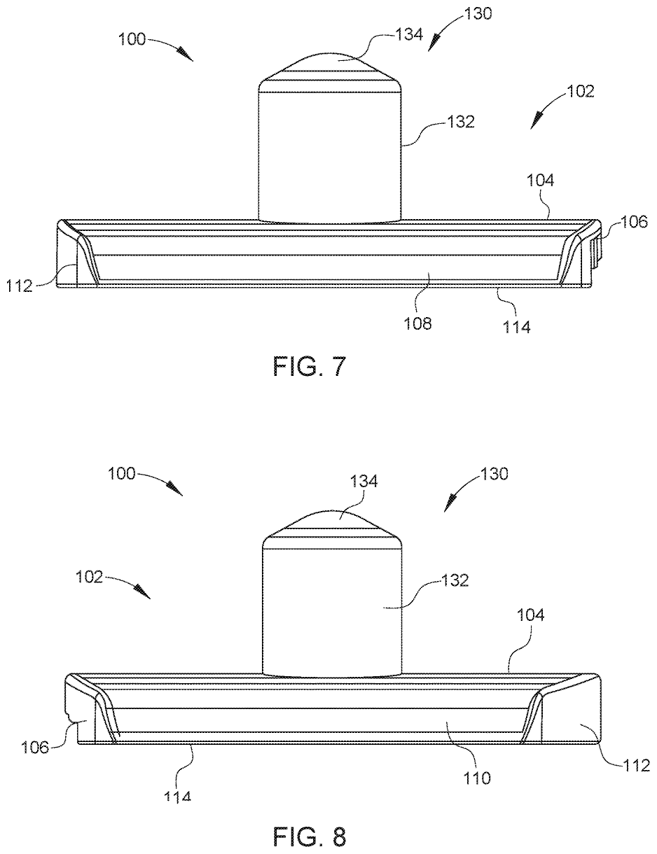

Referring now to FIGS. 1-8, an implant 100 is shown, according to an exemplary embodiment. The implant 100 may be a component of an implant system, for example an ankle arthroplasty (e.g., total ankle replacement or “TAR” as referred to hereinafter) system and as such may include one or more features configured to interface, couple, and/or engage with one or more other components of the system. The implant 100 may also be offered in various sizes and/or dimensions, for example to accommodate various sizes of patient anatomy and/or deformities or conditions with which patients may present. Furthermore, the implant 100 may be offered in configurations where one or more of the components of the implant are variously sized. For example, in some aspects the implant may be offered in configurations with a specific component in small, medium, or large sizes.

The implant 100 includes a body 102, according to the exemplary embodiment of FIGS. 1-8. The body 102 is shown to include a top surface 106 substantially opposite the body 102 from a bottom surface 114, a front end 106 substantially opposite the body 102 from a rear end 112, and a lateral side 108 substantially opposite the body 102 from a medial side 110. As shown, the implant 100 is configured for a right ankle of a patient, where the lateral and medial sides 108, 110 are identified accordingly (and thus would be opposite if the implant 100 were configured for a left ankle of a patient). As shown, the top and bottom surfaces 104, 114 are positioned in planes substantially parallel to one another. Similarly, the lateral and medial sides 108, 110 include respective surfaces at least a portion of which are positioned in planes substantially parallel one another and substantially orthogonal to those of the top and bottom surfaces 104, 114. The front end 106 is shown to include a surface at least a portion of which is positioned in a plane perpendicular to both the planes of the lateral and medial sides 108, 110 and the top and bottom surfaces 104, 114. The rear end 112 is shown to include a curvature (e.g., a radiused portion) along at least a portion thereof, wherein at least a portion of the curved surface thereof is positioned in one or more planes substantially perpendicular to those of the top and bottom surfaces 104, 114.

The body 102 and the top surface 104 thereof are shown to include an extension 130 protruding upward (e.g., substantially perpendicular to at least a portion of the top surface 104) from the top surface 104, at least a portion of which includes a substantially cylindrical geometry. As shown in FIGS. 1-8, the extension 130 is positioned centrally relative to the top surface 104 of the body 102. However, in some aspects the extension 104 may be positioned variously at other locations about the top surface 104 of the implant 100. Further, in some aspects the implant 100 may include one or more extensions 130 positioned variously about the top surface 104 of the implant 100. For example, multiple extensions 130 may be positioned variously about the top surface 104 of the implant 100 so as to accommodate the anatomy of a specific patient (e.g., positioned to fill a distal tibial void or volume created by a cyst or other anatomic condition) and/or positioned to provide additional tibial stability in the distal tibia of a patient upon implantation.

The extension 130 as shown in FIGS. 1-8 is integral with the body 102 and the top surface 104 of the implant 100. However, in some aspects the extension 130 may be releasably couplable or otherwise engageable with the top surface 104 and/or body 102 of the implant 100. For example, a system may be provided to a physician which includes multiple implant bodies 102 and multiple extensions 130, where each of the multiple bodies 102 and extensions 130 are of a different size. Further to the previous example, each of the bodies 102 and extensions 130 may be compatible with one another such that a physician may assemble a desired implant including a desired size body 102 and extension 130 intraoperatively, where the extension 130 is couplable or otherwise engageable with the body 102. In some aspects, the implant 100 may include multiple extension augments, one or more of which may be coupled with the extension 130 and/or other augments so as to adjust (e.g., increase a height of) the extension 130 and dimensions thereof.

The extension 130 includes a shaft 132 which subsequently includes a substantially cylindrical body (e.g., with circular cross-sections and a constant radius/diameter) along at least a portion of the length/height thereof. In some aspects, the extension 130 and/or shaft 132 may include alternate bodies, for example triangular, rectangular, or other geometric prism-shaped geometries. Further, in some aspects the extension 130 and/or shaft 132 may include one or more features disposed on/in the outer surface and/or along a length thereof. For example, the extension 130 and/or shaft 132 may include one or more protrusions, depressions, protuberances, cavities, barbs, or other geometric elements either extending outward or inward from the outer surface thereof. The extension 130 is shown to include a top portion 134 which, as shown, is integral with the shaft 132 but may also be couplable/engageable in some aspects. The top portion 134 is shown to have a substantially semi-spherical or convex geometry including a rounded surface extending in a direction (and including an apex) opposite that of the body 102 relative to the shaft 132. In some aspects, the top portion 134 may include alternate geometries, for example various curvatures/radii of a convex/rounded surfaces, one or more protrusions/barbs/spikes, or other geometric components extending outward or inward from an outer surface thereof.

The bottom surface 114 of the body 102 is shown to include one or more features configured to facilitate releasable coupling and/or engagement with one or more other components of an ankle arthroplasty system (for example, an intermediate component such as a polymeric component including but not limited to those incorporated by reference herein). The bottom surface 114 includes a cavity 116 extending into the body 102 from the bottom surface 114 toward the top surface 104. In some aspects, the cavity 116 may be defined by a continuous, substantially u-shaped perimeter, with at least a portion of the perimeter including an engagement feature 124 (shown in exemplary aspects herein as a female dovetail feature and referred to hereinafter as dovetail 124) configured to facilitate bottom the aforementioned releasable coupling/engagement with one or more other components of an ankle arthroplasty system. The cavity 116 may be positioned such that the cavity 116 is in fluid communication with the front end 106 (e.g., extends into the surface 114/the body 102 from the front end 106 and/or a surface thereof, toward the rear end 112). The bottom surface 114 is further shown to include a cavity 118 and a cavity 120, which may be positioned within the cavity 116 (e.g., extend into the body 102 toward the top surface 106 from within the cavity 116). In some aspects, the cavities 118, 120 may include a substantially rectangular geometry, but may also include other geometries/volumes in alternative aspects. As shown, the cavity 120 is in fluid communication with the front end 106 and is spaced equidistant from opposing sides of the perimeter of the cavity 116. In some aspects, the cavities 116, 118, 120 may be configured such that an intermediate component of an ankle arthroplasty system may be slidably coupled with the implant 100, with the intermediate component positioned adjacent the front end 106 and slid, with an engagement portion (e.g., a male dovetail) aligned with the dovetail 124, toward the rear side 112. Such an intermediate component may also include one or more features configured to releasable couple/engage with one or more of the cavities 118, 120.

The front end 106 of the implant 100 is shown to include a recess 122 disposed on a surface thereof and extending into the body 102 toward the rear side 112. As shown, the recess 122 includes a substantially oval-shaped cross-section, but in some aspects may include other rounded geometric cross-sections or alternate geometric cross-sections. The recess 122 may be configured to engage with (e.g., receive at least a portion thereof within) one or more instruments of an ankle arthroplasty system to facilitate implantation, manipulation, or other various position/repositioning adjustments of the implant 100.

In some aspects, at least a portion of the body 102 and/or the extension 130 may include a textured surface, for example a roughness, configured to facilitate retention of the implant 100 within a distal tibia of a patient. Such a texture may be disposed on at least a portion of the body 102 (e.g., one or more of the top surface 104, the lateral side 108, and the medial side 110) and/or the extension 130 in one or more patterns, according to some aspects. Further, in some aspects the body 102 and/or the extension 130 may include one or more ingrowth surfaces configured to accommodate bony ingrowth and/or packing by a physician with a biologic material.

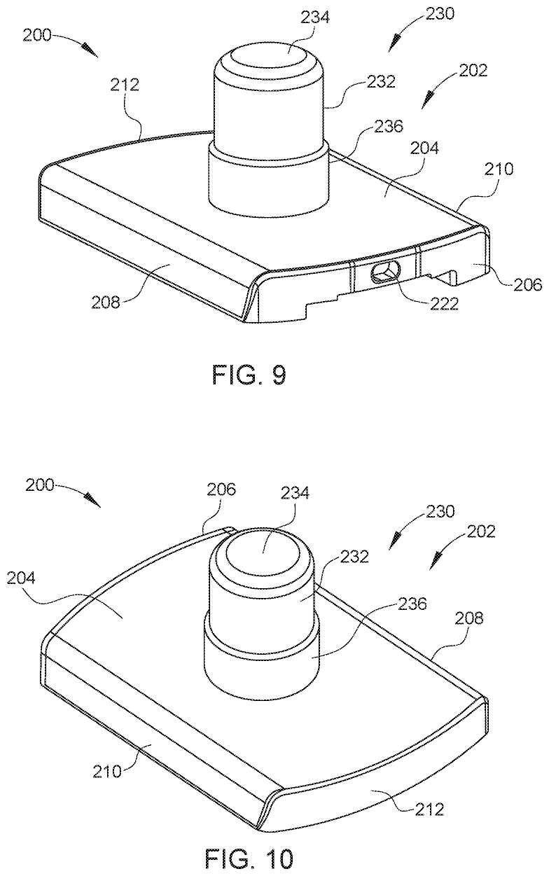

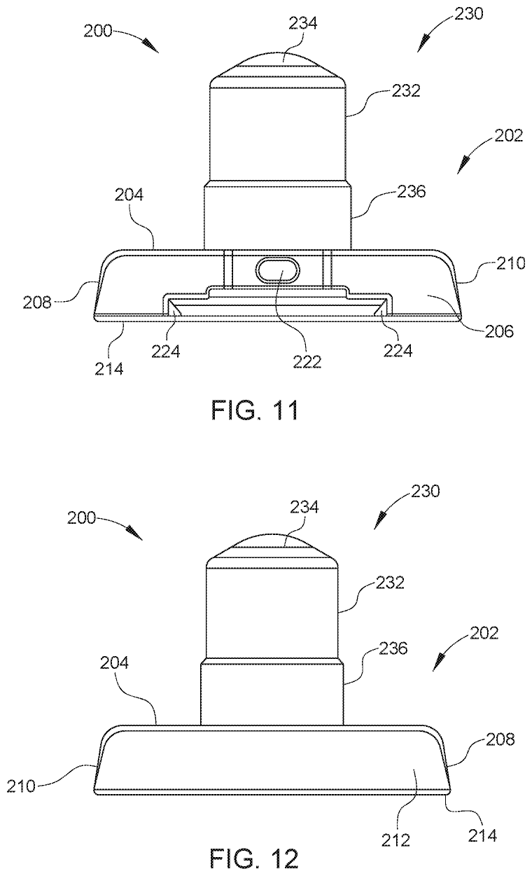

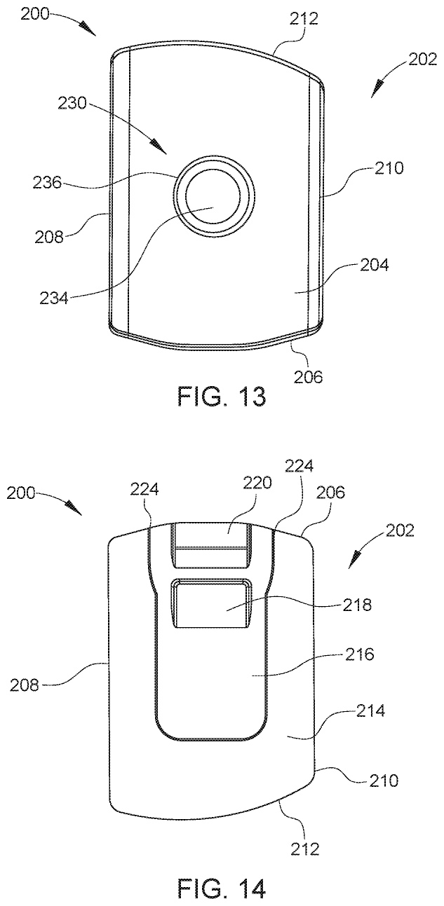

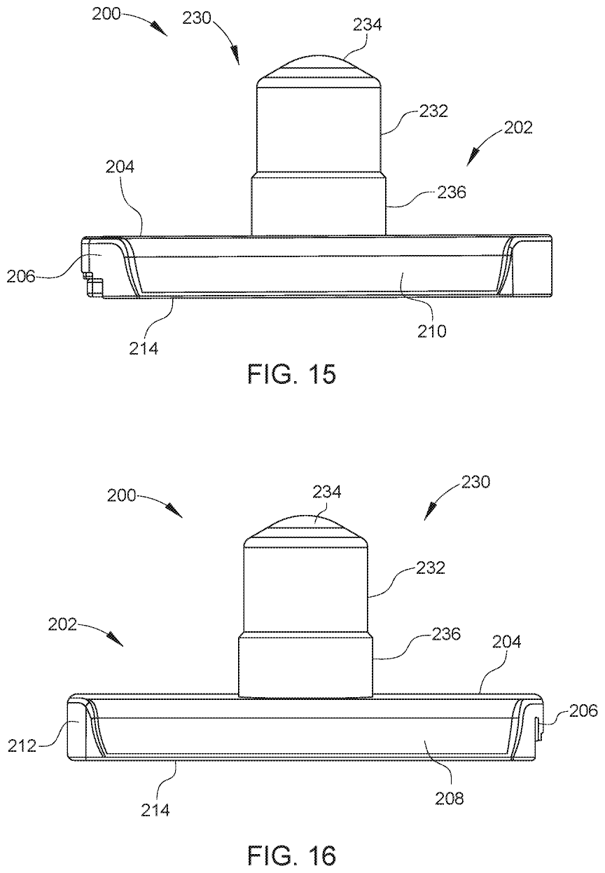

Referring now to FIGS. 9-16, an implant 200 is shown, according to an exemplary embodiment. The implant 200 may be a component of an implant system, for example an ankle arthroplasty or TAR system and as such may include one or more features configured to interface, couple, and/or engage with one or more other components of the system. The implant 200 may also be offered in various sizes and/or dimensions, for example to accommodate various sizes of patient anatomy and/or deformities or conditions with which patients may present. Furthermore, the implant 200 may be offered in configurations where one or more of the components of the implant are variously sized. For example, in some aspects the implant may be offered in configurations with a specific component in small, medium, or large sizes.

The implant 200 includes a body 202, according to the exemplary embodiment of FIGS. 9-16. The body 202 is shown to include a top surface 206 substantially opposite the body 202 from a bottom surface 214, a front end 206 substantially opposite the body 202 from a rear end 212, and a lateral side 208 substantially opposite the body 202 from a medial side 210. As shown, the implant 200 is configured for a right ankle of a patient, where the lateral and medial sides 208, 210 are identified accordingly (and thus would be opposite if the implant 200 were configured for a left ankle of a patient). As shown, the top and bottom surfaces 204, 214 are positioned in planes substantially parallel one another. Similarly, the lateral and medial sides 208, 210 include respective surfaces at least a portion of which are positioned in planes substantially parallel one another and substantially orthogonal to those of the top and bottom surfaces 204, 214. The front end 206 is shown to include a surface at least a portion of which is positioned in a plane perpendicular to both the planes of the lateral and medial sides 208, 210 and the top and bottom surfaces 204, 214. The rear end 212 is shown to include a curvature (e.g., a radiused portion) along at least a portion thereof, wherein at least a portion of the curved surface thereof is positioned in one or more planes substantially perpendicular to those of the top and bottom surfaces 204, 214.

The body 202 and the top surface 204 thereof are shown to include an extension 230 protruding upward (e.g., substantially perpendicular to at least a portion of the top surface 204) from the top surface 204, at least a portion of which includes a substantially cylindrical geometry. As shown in FIGS. 9-16, the extension 230 is positioned centrally relative to the top surface 204 of the body 202. However, in some aspects the extension 204 may be positioned variously at other locations about the top surface 204 of the implant 200. Further, in some aspects the implant 200 may include one or more extensions 230 positioned variously about the top surface 204 of the implant 200. For example, multiple extensions 230 may be positioned variously about the top surface 204 of the implant 200 so as to accommodate the anatomy of a specific patient (e.g., positioned to fill a distal tibial void or volume created by a cyst or other anatomic condition) and/or positioned to provide additional tibial stability in the distal tibia of a patient upon implantation.

The extension 230 as shown in FIGS. 9-16 is integral with the body 202 and the top surface 204 of the implant 200. However, in some aspects the extension 230 may be releasably couplable or otherwise engageable with the top surface 204 and/or body 202 of the implant 200. For example, a system may be provided to a physician which includes multiple implant bodies 202 and multiple extensions 230, where each of the multiple bodies 202 and extensions 230 are of a different size. Further to the previous example, each of the bodies 202 and extensions 230 may be compatible with one another such that a physician may assemble a desired implant including a desired size body 202 and extension 230 intraoperatively, where the extension 230 is couplable or otherwise engageable with the body 202. The extension 230 is shown to include a base 236 which is integral with the body 202 and the extension 230 and includes a geometry that is the same as or similar to that of the extension 230 (for example, a substantially cylindrical shape). As shown, the base 236 includes a greater lateral dimension (as shown, radius/diameter and thus circumference) than that of the extension 230. The base 236 is shown to have a hollow central portion which is occupied by at least a portion of the extension 230, with the base 236 having a height which extends at least a portion of the way up the height of the extension 230. The extension 230 and an outer surface thereof may form a substantially right angle with an upper surface of the base 236, may form a substantially oblique angle with the top surface of the base 236, or include one or more radiused or otherwise curved areas between the top surface of the base 236 and the outer surface of the extension 230. In some aspects, the base 236 may vary in size (e.g., height, diameter/radius, circumference, etc. according to the sizing of respective components of the implant 200. For example, the base 236 may have a greater lateral dimension on a smaller sized implant 200 than a larger size implant 200. Further, the base 236 may be customizable by a physician for a specific size of implant based on the specific anatomy of a patient. In some aspects, the implant 200 may include multiple extension augments, one or more of which may be coupled with the extension 230 and/or other augments so as to adjust (e.g., increase a height of) the extension 230 and dimensions thereof.

The extension 230 includes a shaft 232 which subsequently includes a substantially cylindrical body (e.g., with circular cross-sections and a constant radius/diameter) along at least a portion of the length/height thereof. In some aspects, the extension 230 and/or shaft 232 may include alternate bodies, for example triangular, rectangular, or other geometric prism-shaped geometries. Further, in some aspects the extension 230 and/or shaft 232 may include one or more features disposed on/in the outer surface and/or along a length thereof. For example, the extension 230 and/or shaft 232 may include one or more protrusions, depressions, protuberances, cavities, barbs, or other geometric elements either extending outward or inward from the outer surface thereof. The extension 230 is shown to include a top portion 234 which, as shown, is integral with the shaft 232 but may also be couplable/engageable in some aspects. The top portion 234 is shown to have a substantially semi-spherical or convex geometry including a rounded surface extending in a direction (and including an apex) opposite that of the body 202 relative to the shaft 232. In some aspects, the top portion 234 may include alternate geometries, for example various curvatures/radii of a convex/rounded surfaces, one or more protrusions/barbs/spikes, or other geometric components extending outward or inward from an outer surface thereof.

The bottom surface 214 of the body 202 is shown to include one or more features configured to facilitate releasable coupling and/or engagement with one or more other components of an ankle arthroplasty system (for example, an intermediate component such as a polymeric component including but not limited to those incorporated by reference herein). The bottom surface 214 includes a cavity 216 extending into the body 202 from the bottom surface 214 toward the top surface 204. In some aspects, the cavity 216 may be defined by a continuous, substantially u-shaped perimeter, with at least a portion of the perimeter including an engagement feature 224 (shown in exemplary aspects herein as a female dovetail feature and referred to hereinafter as dovetail 224) configured to facilitate bottom the aforementioned releasable coupling/engagement with one or more other components of an ankle arthroplasty system. The cavity 216 may be positioned such that the cavity 216 is in fluid communication with the front end 206 (e.g., extends into the surface 214/the body 202 from the front end 206 and/or a surface thereof, toward the rear end 212). The bottom surface 214 is further shown to include a cavity 218 and a cavity 220, which may be positioned within the cavity 216 (e.g., extend into the body 202 toward the top surface 206 from within the cavity 216). In some aspects, the cavities 218, 220 may include a substantially rectangular geometry, but may also include other geometries/volumes in alternative aspects. As shown, the cavity 220 is in fluid communication with the front end 206 and is spaced equidistant from opposing sides of the perimeter of the cavity 216. In some aspects, the cavities 216, 218, 220 may be configured such that an intermediate component of an ankle arthroplasty system may be slidably coupled with the implant 200, with the intermediate component positioned adjacent the front end 206 and slid, with an engagement portion (e.g., a male dovetail) aligned with the dovetail 224, toward the rear side 212. Such an intermediate component may also include one or more features configured to releasable couple/engage with one or more of the cavities 218, 220.

The front end 206 of the implant 200 is shown to include a recess 222 disposed on a surface thereof and extending into the body 202 toward the rear side 212. As shown, the recess 222 includes a substantially oval-shaped cross-section, but in some aspects may include other rounded geometric cross-sections or alternate geometric cross-sections. The recess 222 may be configured to engage with (e.g., receive at least a portion thereof within) one or more instruments of an ankle arthroplasty system to facilitate implantation, manipulation, or other various position/repositioning adjustments of the implant 200.

In some aspects, at least a portion of the body 202 and/or the extension 230 may include a textured surface, for example a roughness, configured to facilitate retention of the implant 200 within a distal tibia of a patient. Such a textured surface may be disposed on at least a portion of the body 202 (e.g., one or more of the top surface 204, the lateral side 208, the medial side 210, and the base 236) and/or the extension 230 in one or more patterns, according to some aspects. Further, in some aspects the body 202 and/or the extension 230 may include one or more ingrowth surfaces configured to accommodate bony ingrowth and/or packing by a physician with a biologic material.

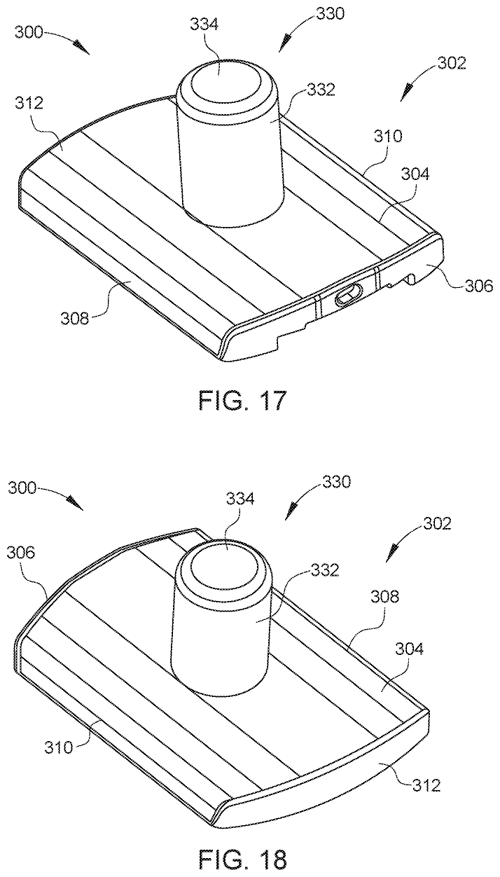

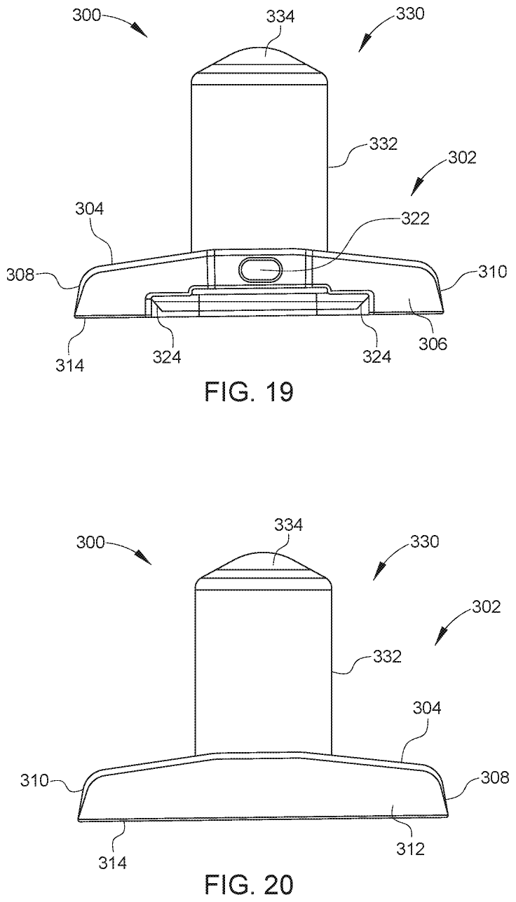

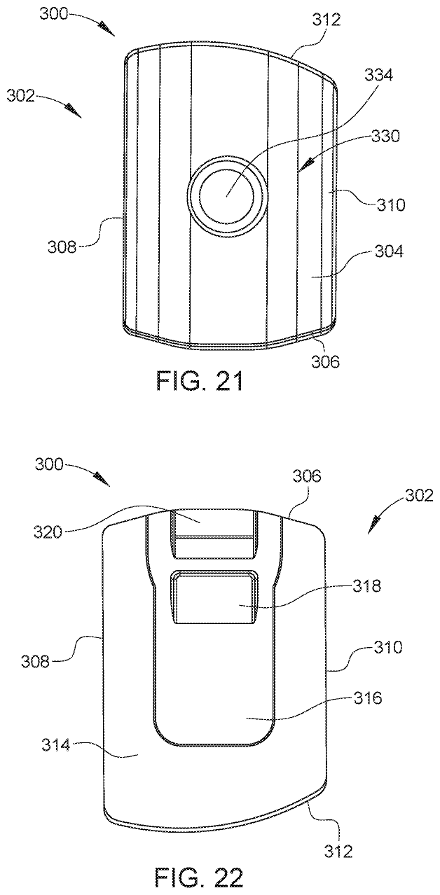

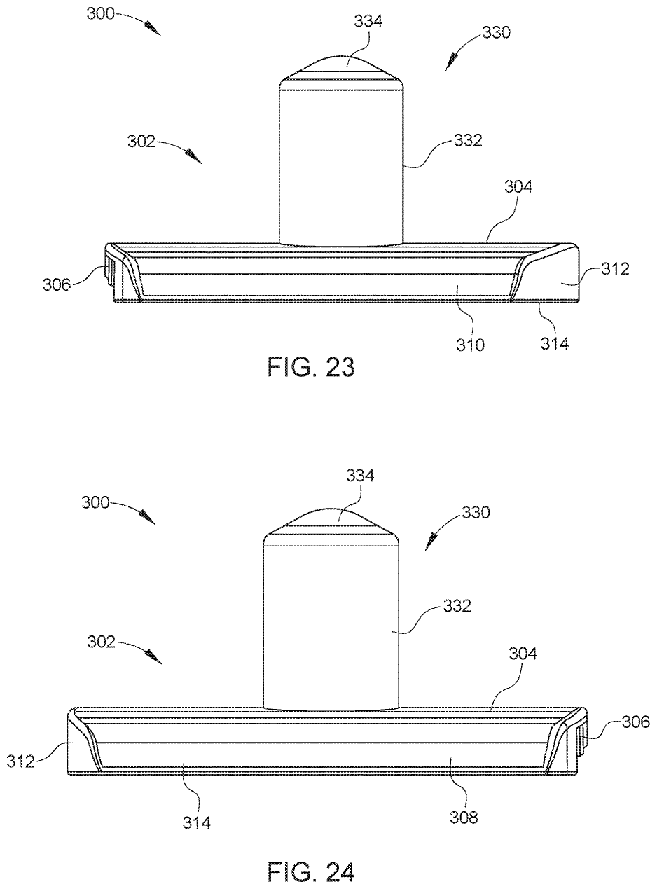

Referring now to FIGS. 17-24, an implant 300 is shown, according to an exemplary embodiment. The implant 300 may be a component of an implant system, for example an ankle arthroplasty or TAR system and as such may include one or more features configured to interface, couple, and/or engage with one or more other components of the system. The implant 300 may also be offered in various sizes and/or dimensions, for example to accommodate various sizes of patient anatomy and/or deformities or conditions with which patients may present. Furthermore, the implant 300 may be offered in configurations where one or more of the components of the implant are variously sized. For example, in some aspects the implant may be offered in configurations with a specific component in small, medium, or large sizes.

The implant 300 includes a body 302, according to the exemplary embodiment of FIGS. 17-24. The body 302 is shown to include a top surface 306 substantially opposite the body 302 from a bottom surface 314, a front end 306 substantially opposite the body 302 from a rear end 312, and a lateral side 308 substantially opposite the body 302 from a medial side 310. As shown, the implant 300 is configured for a right ankle of a patient, where the lateral and medial sides 308, 310 are identified accordingly (and thus would be opposite if the implant 300 were configured for a left ankle of a patient). As shown, the top and bottom surfaces 304, 314 are positioned in planes substantially parallel one another. Similarly, the lateral and medial sides 308, 310 include respective surfaces at least a portion of which are positioned in planes substantially parallel one another and substantially orthogonal to those of the top and bottom surfaces 304, 314. The front end 306 is shown to include a surface at least a portion of which is positioned in a plane perpendicular to both the planes of the lateral and medial sides 308, 310 and the top and bottom surfaces 304, 314. The rear end 312 is shown to include a curvature (e.g., a radiused portion) along at least a portion thereof, wherein at least a portion of the curved surface thereof is positioned in one or more planes substantially perpendicular to those of the top and bottom surfaces 304, 314.

The body 302 and the top surface 304 thereof are shown to include an extension 330 protruding upward (e.g., substantially perpendicular to at least a portion of the top surface 304) from the top surface 304, at least a portion of which includes a substantially cylindrical geometry. As shown in FIGS. 17-24, the extension 330 is positioned centrally relative to the top surface 304 of the body 302. However, in some aspects the extension 304 may be positioned variously at other locations about the top surface 304 of the implant 300. Further, in some aspects the implant 300 may include one or more extensions 330 positioned variously about the top surface 304 of the implant 300. For example, multiple extension 330 may be positioned variously about the top surface 304 of the implant 300 so as to accommodate the anatomy of a specific patient (e.g., positioned to fill a distal tibial void or volume created by a cyst or other anatomic condition) and/or positioned to provide additional tibial stability in the distal tibia of a patient upon implantation.

The extension 330 as shown in FIGS. 17-24 is integral with the body 302 and the top surface 304 of the implant 300. However, in some aspects the extension 330 may be releasably couplable or otherwise engageable with the top surface 304 and/or body 302 of the implant 300. For example, a system may be provided to a physician which includes multiple implant bodies 302 and multiple extensions 330, where each of the multiple bodies 302 and extensions 330 are of a different size. Further to the previous example, each of the bodies 302 and extensions 330 may be compatible with one another such that a physician may assemble a desired implant including a desired size body 302 and extension 330 intraoperatively, where the extension 330 is couplable or otherwise engageable with the body 302. The extension 330 may be similar in geometry to that of the extension 130 of FIGS. 1-8, but may also have a greater height (e.g., 12 mm height vs. 15 mm height on the extension 330) so as to extend further proximally into the distal tibia upon implantation. In some aspects, the implant 300 may include multiple extension augments, one or more of which may be coupled with the extension 330 and/or other augments so as to adjust (e.g., increase a height of) the extension 330 and dimensions thereof.

The extension 330 includes a shaft 332 which subsequently includes a substantially cylindrical body (e.g., with circular cross-sections and a constant radius/diameter) along at least a portion of the length/height thereof. In some aspects, the extension 330 and/or shaft 332 may include alternate bodies, for example triangular, rectangular, or other geometric prism-shaped geometries. Further, in some aspects the extension 330 and/or shaft 332 may include one or more features disposed on/in the outer surface and/or along a length thereof. For example, the extension 330 and/or shaft 332 may include one or more protrusions, depressions, protuberances, cavities, barbs, or other geometric elements either extending outward or inward from the outer surface thereof. The extension 330 is shown to include a top portion 334 which, as shown, is integral with the shaft 332 but may also be couplable/engageable in some aspects. The top portion 334 is shown to have a substantially semi-spherical or convex geometry including a rounded surface extending in a direction (and including an apex) opposite that of the body 302 relative to the shaft 332. In some aspects, the top portion 334 may include alternate geometries, for example various curvatures/radii of a convex/rounded surfaces, one or more protrusions/barbs/spikes, or other geometric components extending outward or inward from an outer surface thereof.

The bottom surface 314 of the body 302 is shown to include one or more features configured to facilitate releasable coupling and/or engagement with one or more other components of an ankle arthroplasty system (for example, an intermediate component such as a polymeric component, including but not limited to, those incorporated by reference herein). The bottom surface 314 includes a cavity 316 extending into the body 302 from the bottom surface 314 toward the top surface 304. In some aspects, the cavity 316 may be defined by a continuous, substantially u-shaped perimeter, with at least a portion of the perimeter including an engagement feature 324 (shown in exemplary aspects herein as a female dovetail feature and referred to hereinafter as dovetail 324) configured to facilitate bottom the aforementioned releasable coupling/engagement with one or more other components of an ankle arthroplasty system. The cavity 316 may be positioned such that the cavity 316 is in fluid communication with the front end 306 (e.g., extends into the surface 314/the body 302 from the front end 306 and/or a surface thereof, toward the rear end 312). The bottom surface 314 is further shown to include a cavity 318 and a cavity 320, which may be positioned within the cavity 316 (e.g., extend into the body 302 toward the top surface 306 from within the cavity 316). In some aspects, the cavities 318, 320 may include a substantially rectangular geometry, but may also include other geometries/volumes in alternative aspects. As shown, the cavity 320 is in fluid communication with the front end 306 and is spaced equidistant from opposing sides of the perimeter of the cavity 316. In some aspects, the cavities 316, 318, 320 may be configured such that an intermediate component of an ankle arthroplasty system may be slidably coupled with the implant 300, with the intermediate component positioned adjacent the front end 306 and slid, with an engagement portion (e.g., a male dovetail) aligned with the dovetail 324, toward the rear side 312. Such an intermediate component may also include one or more features configured to releasable couple/engage with one or more of the cavities 318, 320.

The front end 306 of the implant 300 is shown to include a recess 322 disposed on a surface thereof and extending into the body 302 toward the rear side 312. As shown, the recess 322 includes a substantially oval-shaped cross-section, but in some aspects may include other rounded geometric cross-sections or alternate geometric cross-sections. The recess 322 may be configured to engage with (e.g., receive at least a portion thereof within) one or more instruments of an ankle arthroplasty system to facilitate implantation, manipulation, or other various position/repositioning adjustments of the implant 300.

In some aspects, at least a portion of the body 302 and/or the extension 330 may include a textured surface, for example a roughness, configured to facilitate retention of the implant 300 within a distal tibia of a patient. Such a textured surface may be disposed on at least a portion of the body 302 (e.g., one or more of the top surface 304, the lateral side 308, the medial side 310) and/or the extension 330 in one or more patterns, according to some aspects. Further, in some aspects the body 302 and/or the extension 330 may include one or more ingrowth surfaces configured to accommodate bony ingrowth and/or packing by a physician with a biologic material.

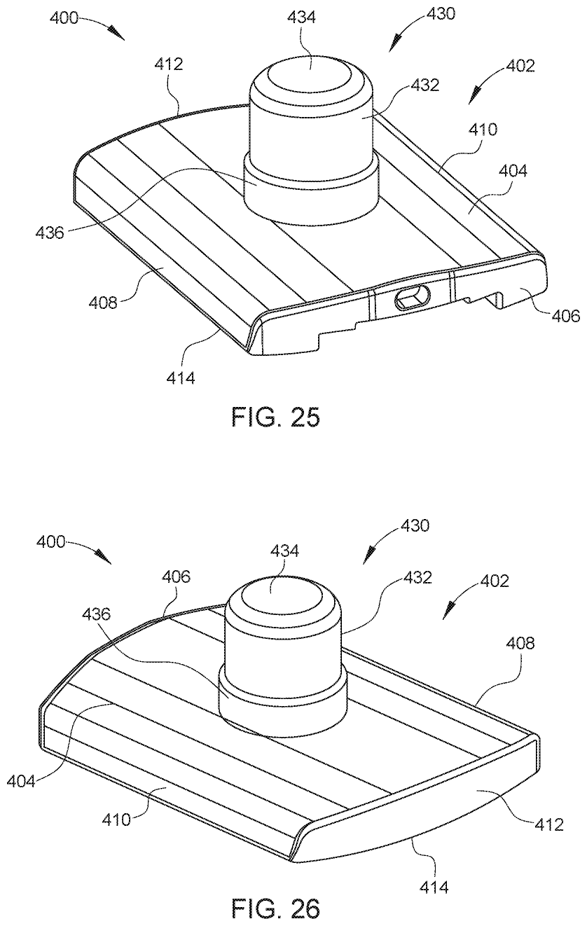

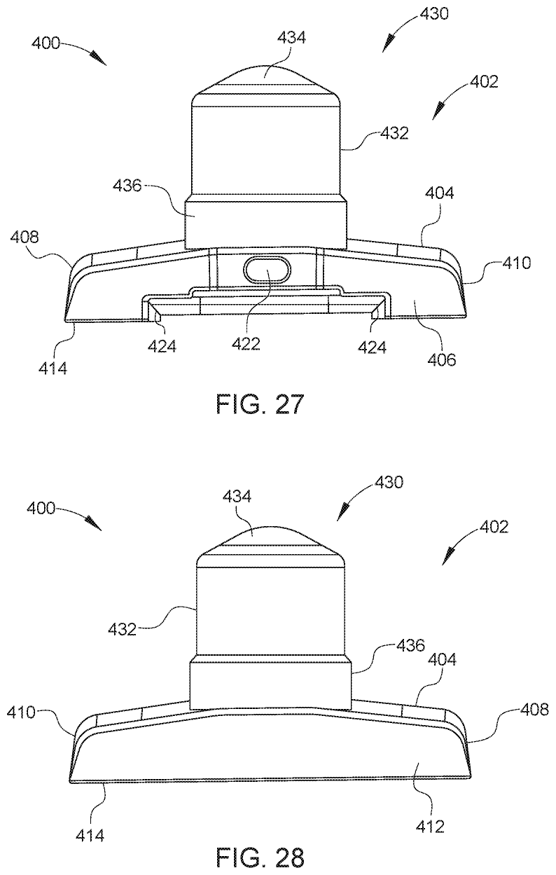

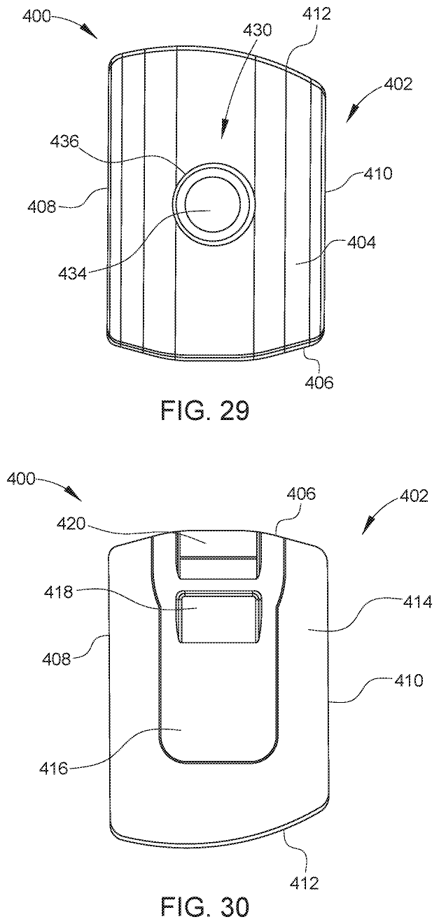

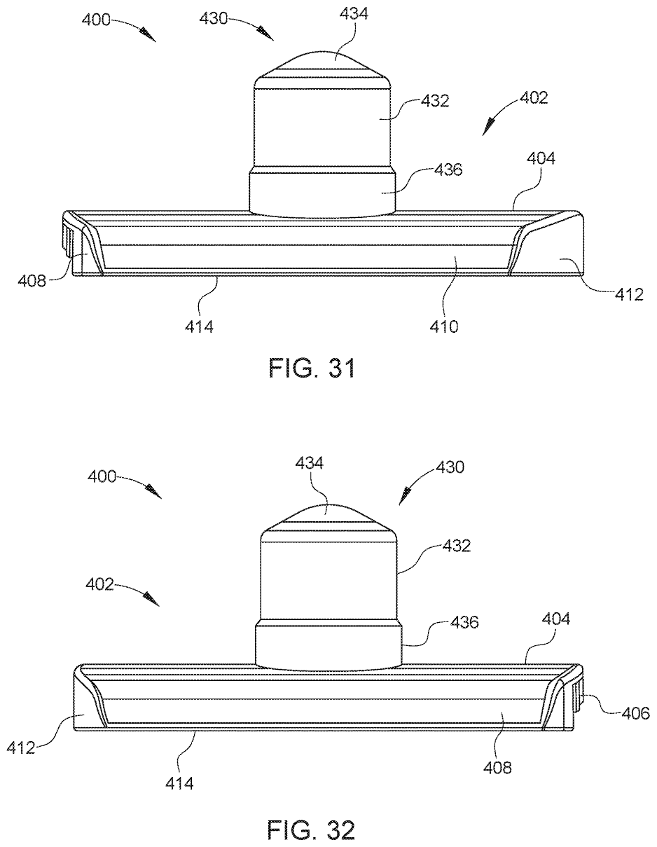

Referring now to FIGS. 25-32, an implant 400 is shown, according to an exemplary embodiment. The implant 400 may be a component of an implant system, for example an ankle arthroplasty or TAR system and as such may include one or more features configured to interface, couple, and/or engage with one or more other components of the system. The implant 400 may also be offered in various sizes and/or dimensions, for example to accommodate various sizes of patient anatomy and/or deformities or conditions with which patients may present. Furthermore, the implant 400 may be offered in configurations where one or more of the components of the implant are variously sized. For example, in some aspects the implant may be offered in configurations with a specific component in small, medium, or large sizes.

The implant 400 includes a body 402, according to the exemplary embodiment shown in FIGS. 25-32. The body 402 is shown to include a top surface 406 substantially opposite the body 402 from a bottom surface 414, a front end 406 substantially opposite the body 402 from a rear end 412, and a lateral side 408 substantially opposite the body 402 from a medial side 410. As shown, the implant 400 is configured for a right ankle of a patient, where the lateral and medial sides 408, 410 are identified accordingly (and thus would be opposite if the implant 400 were configured for a left ankle of a patient). As shown, the top and bottom surfaces 404, 414 are positioned in planes substantially parallel to one another. Similarly, the lateral and medial sides 408, 410 include respective surfaces at least a portion of which are positioned in planes substantially parallel to one another and substantially orthogonal to those of the top and bottom surfaces 404, 414. The front end 406 is shown to include a surface at least a portion of which is positioned in a plane perpendicular to both the planes of the lateral and medial sides 408, 410 and the top and bottom surfaces 404, 414. The rear end 412 is shown to include a curvature (e.g., a radiused portion) along at least a portion thereof, wherein at least a portion of the curved surface thereof is positioned in one or more planes substantially perpendicular to those of the top and bottom surfaces 404, 414.

The body 402 and the top surface 404 thereof are shown to include an extension 430 protruding upward (e.g., substantially perpendicular to at least a portion of the top surface 404) from the top surface 404, at least a portion of which includes a substantially cylindrical geometry. As shown in FIGS. 25-32, the extension 430 is positioned centrally relative to the top surface 404 of the body 402. However, in some aspects the extension 404 may be positioned variously at other locations about the top surface 404 of the implant 400. Further, in some aspects the implant 400 may include one or more extensions 430 positioned variously about the top surface 404 of the implant 400. For example, multiple extensions 430 may be positioned variously about the top surface 404 of the implant 400 so as to accommodate the anatomy of a specific patient (e.g., positioned to fill a distal tibial void or volume created by a cyst or other anatomic condition) and/or positioned to provide additional tibial stability in the distal tibia of a patient upon implantation.

The extension 430 as shown in FIGS. 25-32 is integral with the body 402 and the top surface 404 of the implant 400. However, in some aspects the extension 430 may be releasably couplable or otherwise engageable with the top surface 404 and/or body 402 of the implant 400. For example, a system may be provided to a physician which includes multiple implant bodies 402 and multiple extensions 430, where each of the multiple bodies 402 and extensions 430 are of a different size. Further to the previous example, each of the bodies 402 and extensions 430 may be compatible with one another such that a physician may assemble a desired implant including a desired size body 402 and extension 430 intraoperatively, where the extension 430 is couplable or otherwise engageable with the body 402. The extension 430 is shown to include a base 436 which is integral with the body 402 and the extension 430 and includes a geometry that is the same as or similar to that of the extension 430 (for example, a substantially cylindrical shape). As shown, the base 436 includes a greater lateral dimension (as shown, radius/diameter and thus circumference) than that of the extension 430. The base 436 is shown to have a hollow central portion which is occupied by at least a portion of the extension 430, with the base 436 having a height which extends at least a portion of the way up the height of the extension 430. The extension 430 and an outer surface thereof may form a substantially right angle with an upper surface of the base 436, may form a substantially oblique angle with the top surface of the base 436, or include one or more radiused or otherwise curved areas between the top surface of the base 436 and the outer surface of the extension 430. In some aspects, the base 436 may vary in size (e.g., height, diameter/radius, circumference, etc. according to the sizing of respective components of the implant 400. For example, the base 436 may have a greater lateral dimension on a smaller sized implant 400 than a larger size implant 400. Further, the base 436 may be customizable by a physician for a specific size of implant based on the specific anatomy of a patient. The extension 430 may be similar in geometry to that of the extension 230 of FIGS. 9-16, but may also have a lesser height (e.g., a 12 mm height vs. 15 mm height on the extension 230) so as to extend further proximally into the distal tibia upon implantation. In some aspects, the implant 400 may include multiple extension augments, one or more of which may be coupled with the extension 430 and/or other augments so as to adjust (e.g., increase a height of) the extension 430 and dimensions thereof.

The extension 430 includes a shaft 432 which subsequently includes a substantially cylindrical body (e.g., with circular cross-sections and a constant radius/diameter) along at least a portion of the length/height thereof. In some aspects, the extension 430 and/or shaft 432 may include alternate bodies, for example triangular, rectangular, or other geometric prism-shaped geometries. Further, in some aspects the extension 430 and/or shaft 432 may include one or more features disposed on/in the outer surface and/or along a length thereof. For example, the extension 430 and/or shaft 432 may include one or more protrusions, depressions, protuberances, cavities, barbs, or other geometric elements either extending outward or inward from the outer surface thereof. The extension 430 is shown to include a top portion 434 which, as shown, is integral with the shaft 432 but may also be couplable/engageable in some aspects. The top portion 434 is shown to have a substantially semi-spherical or convex geometry including a rounded surface extending in a direction (and including an apex) opposite that of the body 402 relative to the shaft 432. In some aspects, the top portion 434 may include alternate geometries, for example various curvatures/radii of a convex/rounded surfaces, one or more protrusions/barbs/spikes, or other geometric components extending outward or inward from an outer surface thereof.

The bottom surface 414 of the body 402 is shown to include one or more features configured to facilitate releasable coupling and/or engagement with one or more other components of an ankle arthroplasty system (for example, an intermediate component such as a polymeric component including but not limited to those incorporated by reference herein). The bottom surface 414 includes a cavity 416 extending into the body 402 from the bottom surface 414 toward the top surface 404. In some aspects, the cavity 416 may be defined by a continuous, substantially u-shaped perimeter, with at least a portion of the perimeter including an engagement feature 424 (shown in exemplary aspects herein as a female dovetail feature and referred to hereinafter as dovetail 424) configured to facilitate bottom the aforementioned releasable coupling/engagement with one or more other components of an ankle arthroplasty system. The cavity 416 may be positioned such that the cavity 416 is in fluid communication with the front end 406 (e.g., extends into the surface 414/the body 402 from the front end 406 and/or a surface thereof, toward the rear end 412). The bottom surface 414 is further shown to include a cavity 418 and a cavity 420, which may be positioned within the cavity 416 (e.g., extend into the body 402 toward the top surface 406 from within the cavity 416). In some aspects, the cavities 418, 420 may include a substantially rectangular geometry, but may also include other geometries/volumes in alternative aspects. As shown, the cavity 420 is in fluid communication with the front end 406 and is spaced equidistant from opposing sides of the perimeter of the cavity 416. In some aspects, the cavities 416, 418, 420 may be configured such that an intermediate component of an ankle arthroplasty system may be slidably coupled with the implant 400, with the intermediate component positioned adjacent the front end 406 and slid, with an engagement portion (e.g., a male dovetail) aligned with the dovetail 424, toward the rear side 412. Such an intermediate component may also include one or more features configured to releasable couple/engage with one or more of the cavities 418, 420.

The front end 406 of the implant 400 is shown to include a recess 422 disposed on a surface thereof and extending into the body 402 toward the rear side 412. As shown, the recess 422 includes a substantially oval-shaped cross-section, but in some aspects may include other rounded geometric cross-sections or alternate geometric cross-sections. The recess 422 may be configured to engage with (e.g., receive at least a portion thereof within) one or more instruments of an ankle arthroplasty system to facilitate implantation, manipulation, or other various position/repositioning adjustments of the implant 400.

In some aspects, at least a portion of the body 402 and/or the extension 430 may include a textured surface, for example a roughness, configured to facilitate retention of the implant 400 within a distal tibia of a patient. Such a textured surface may be disposed on at least a portion of the body 402 (e.g., one or more of the top surface 404, the lateral side 408, the medial side 410, and the base 436) and/or the extension 430 in one or more patterns, according to some aspects. Further, in some aspects the body 402 and/or the extension 430 may include one or more ingrowth surfaces configured to accommodate bony ingrowth and/or packing by a physician with a biologic material.

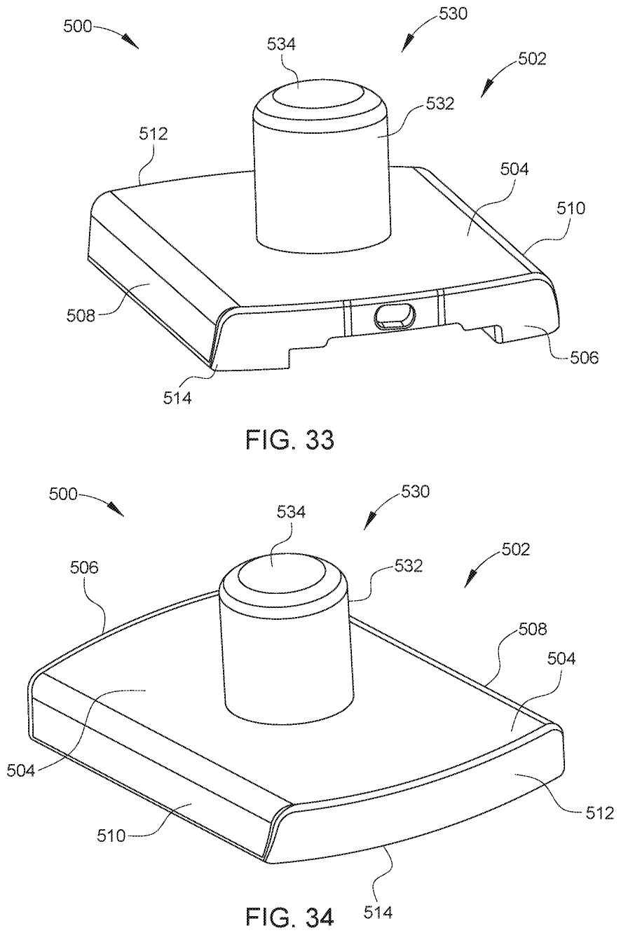

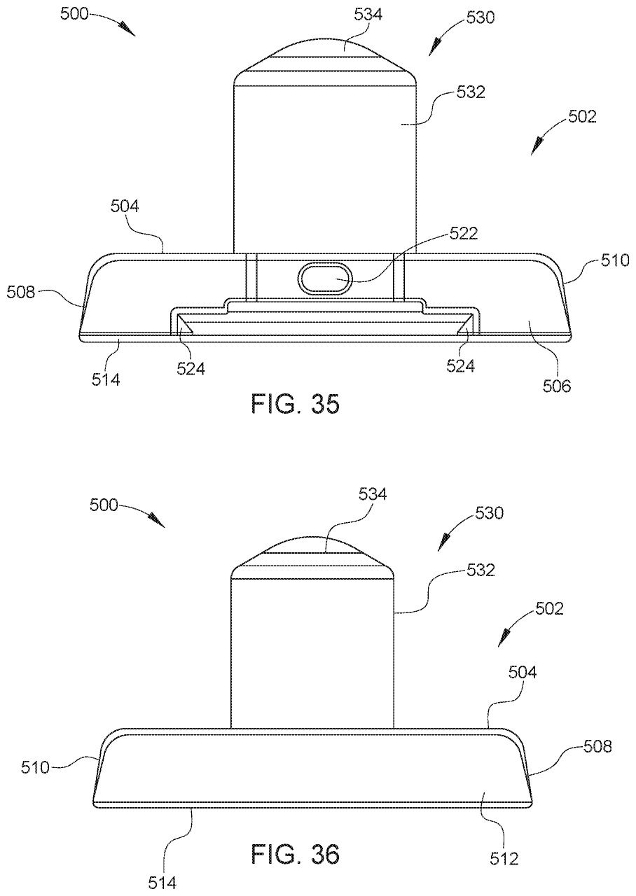

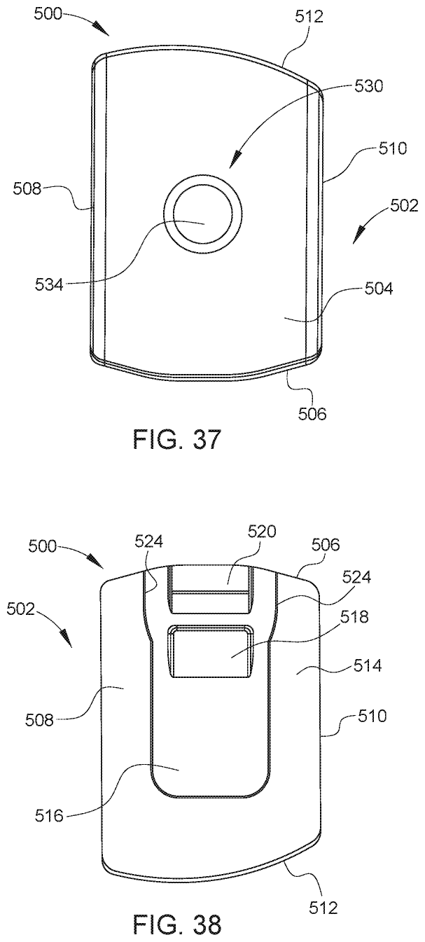

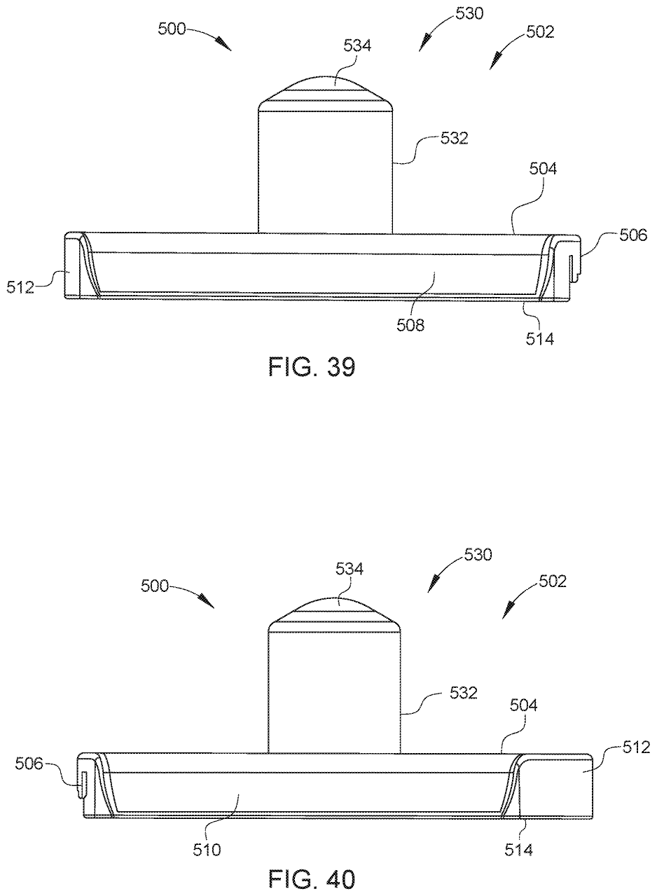

Referring now to FIGS. 33-40, an implant 500 is shown, according to an exemplary embodiment. The implant 500 may be a component of an implant system, for example an ankle arthroplasty or TAR system and as such may include one or more features configured to interface, couple, and/or engage with one or more other components of the system. The implant 500 may also be offered in various sizes and/or dimensions, for example to accommodate various sizes of patient anatomy and/or deformities or conditions with which patients may present. Furthermore, the implant 500 may be offered in configurations where one or more of the components of the implant are variously sized. For example, in some aspects the implant may be offered in configurations with a specific component in small, medium, or large sizes.

The implant 500 includes a body 502, according to the exemplary embodiment of FIGS. 33-40. The body 502 is shown to include a top surface 506 substantially opposite the body 502 from a bottom surface 514, a front end 506 substantially opposite the body 502 from a rear end 512, and a lateral side 508 substantially opposite the body 502 from a medial side 510. As shown, the implant 500 is configured for a right ankle of a patient, where the lateral and medial sides 508, 510 are identified accordingly (and thus would be opposite if the implant 500 were configured for a left ankle of a patient). As shown, the top and bottom surfaces 504, 514 are positioned in planes substantially parallel to one another. Similarly, the lateral and medial sides 508, 510 include respective surfaces at least a portion of which are positioned in planes substantially parallel to one another and substantially orthogonal to those of the top and bottom surfaces 504, 514. The front end 506 is shown to include a surface at least a portion of which is positioned in a plane perpendicular to both the planes of the lateral and medial sides 508, 510 and the top and bottom surfaces 504, 514. The rear end 512 is shown to include a curvature (e.g., a radiused portion) along at least a portion thereof, wherein at least a portion of the curved surface thereof is positioned in one or more planes substantially perpendicular to those of the top and bottom surfaces 504, 514.

The body 502 and the top surface 504 thereof are shown to include an extension 530 protruding upward (e.g., substantially perpendicular to at least a portion of the top surface 504) from the top surface 504, at least a portion of which includes a substantially cylindrical geometry. As shown in FIGS. 33-40, the extension 530 is positioned centrally relative to the top surface 504 of the body 502. However, in some aspects the extension 504 may be positioned variously at other locations about the top surface 504 of the implant 500. Further, in some aspects the implant 500 may include one or more extensions 530 positioned variously about the top surface 504 of the implant 500. For example, multiple extension 530 may be positioned variously about the top surface 504 of the implant 500 so as to accommodate the anatomy of a specific patient (e.g., positioned to fill a distal tibial void or volume created by a cyst or other anatomic condition) and/or positioned to provide additional tibial stability in the distal tibia of a patient upon implantation.

The extension 530 as shown in FIGS. 33-40 is integral with the body 502 and the top surface 504 of the implant 500. However, in some aspects the extension 530 may be releasably couplable or otherwise engageable with the top surface 504 and/or body 502 of the implant 500. For example, a system may be provided to a physician which includes multiple implant bodies 502 and multiple extensions 530, where each of the multiple bodies 502 and extensions 530 are of a different size. Further to the previous example, each of the bodies 502 and extensions 530 may be compatible with one another such that a physician may assemble a desired implant including a desired size body 502 and extension 530 intraoperatively, where the extension 530 is couplable or otherwise engageable with the body 502. The extension 530 may be similar in geometry to that of the extension 130 of FIGS. 1-8 and/or 330 of FIGS. 17-24, but may also have a greater height (e.g., 12 mm height vs. 15 mm height on the extension 130) so as to extend further proximally into the distal tibia upon implantation. In some aspects, the implant 500 may include multiple extension augments, one or more of which may be coupled with the extension 530 and/or other augments so as to adjust (e.g., increase a height of) the extension 530 and dimensions thereof.

The extension 530 includes a shaft 532 which subsequently includes a substantially cylindrical body (e.g., with circular cross-sections and a constant radius/diameter) along at least a portion of the length/height thereof. In some aspects, the extension 530 and/or shaft 532 may include alternate bodies, for example triangular, rectangular, or other geometric prism-shaped geometries. Further, in some aspects the extension 530 and/or shaft 532 may include one or more features disposed on/in the outer surface and/or along a length thereof. For example, the extension 530 and/or shaft 532 may include one or more protrusions, depressions, protuberances, cavities, barbs, or other geometric elements either extending outward or inward from the outer surface thereof. The extension 530 is shown to include a top portion 534 which, as shown, is integral with the shaft 532 but may also be couplable/engageable in some aspects. The top portion 534 is shown to have a substantially semi-spherical or convex geometry including a rounded surface extending in a direction (and including an apex) opposite that of the body 502 relative to the shaft 532. In some aspects, the top portion 534 may include alternate geometries, for example various curvatures/radii of a convex/rounded surfaces, one or more protrusions/barbs/spikes, or other geometric components extending outward or inward from an outer surface thereof.

The bottom surface 514 of the body 502 is shown to include one or more features configured to facilitate releasable coupling and/or engagement with one or more other components of an ankle arthroplasty system (for example, an intermediate component such as a polymeric component, including but not limited to, those incorporated by reference herein). The bottom surface 514 includes a cavity 516 extending into the body 502 from the bottom surface 514 toward the top surface 504. In some aspects, the cavity 516 may be defined by a continuous, substantially u-shaped perimeter, with at least a portion of the perimeter including an engagement feature 524 (shown in exemplary aspects herein as a female dovetail feature and referred to hereinafter as dovetail 524) configured to facilitate bottom the aforementioned releasable coupling/engagement with one or more other components of an ankle arthroplasty system. The cavity 516 may be positioned such that the cavity 516 is in fluid communication with the front end 506 (e.g., extends into the surface 514/the body 502 from the front end 506 and/or a surface thereof, toward the rear end 512). The bottom surface 514 is further shown to include a cavity 518 and a cavity 520, which may be positioned within the cavity 516 (e.g., extend into the body 502 toward the top surface 506 from within the cavity 516). In some aspects, the cavities 518, 520 may include a substantially rectangular geometry, but may also include other geometries/volumes in alternative aspects. As shown, the cavity 520 is in fluid communication with the front end 506 and is spaced equidistant from opposing sides of the perimeter of the cavity 516. In some aspects, the cavities 516, 518, 520 may be configured such that an intermediate component of an ankle arthroplasty system may be slidably coupled with the implant 500, with the intermediate component positioned adjacent the front end 506 and slid, with an engagement portion (e.g., a male dovetail) aligned with the dovetail 524, toward the rear side 512. Such an intermediate component may also include one or more features configured to releasable couple/engage with one or more of the cavities 518, 520.

The front end 506 of the implant 500 is shown to include a recess 522 disposed on a surface thereof and extending into the body 502 toward the rear side 512. As shown, the recess 522 includes a substantially oval-shaped cross-section, but in some aspects may include other rounded geometric cross-sections or alternate geometric cross-sections. The recess 522 may be configured to engage with (e.g., receive at least a portion thereof within) one or more instruments of an ankle arthroplasty system to facilitate implantation, manipulation, or other various position/repositioning adjustments of the implant 500.

In some aspects, at least a portion of the body 502 and/or the extension 530 may include a textured surface, for example a roughness, configured to facilitate retention of the implant 500 within a distal tibia of a patient. Such a textured surface may be disposed on at least a portion of the body 502 (e.g., one or more of the top surface 504, the lateral side 508, the medial side 510) and/or the extension 530 in one or more patterns, according to some aspects. Further, in some aspects the body 502 and/or the extension 530 may include one or more ingrowth surfaces configured to accommodate bony ingrowth and/or packing by a physician with a biologic material.

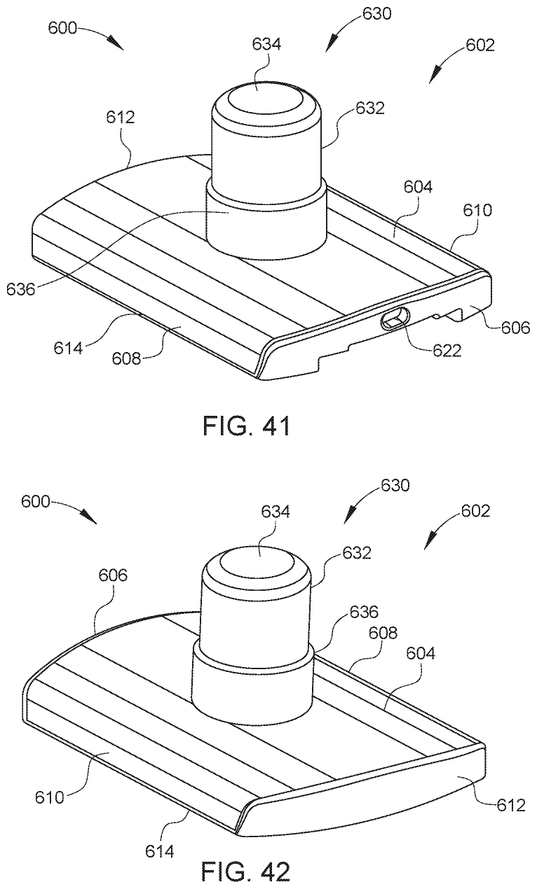

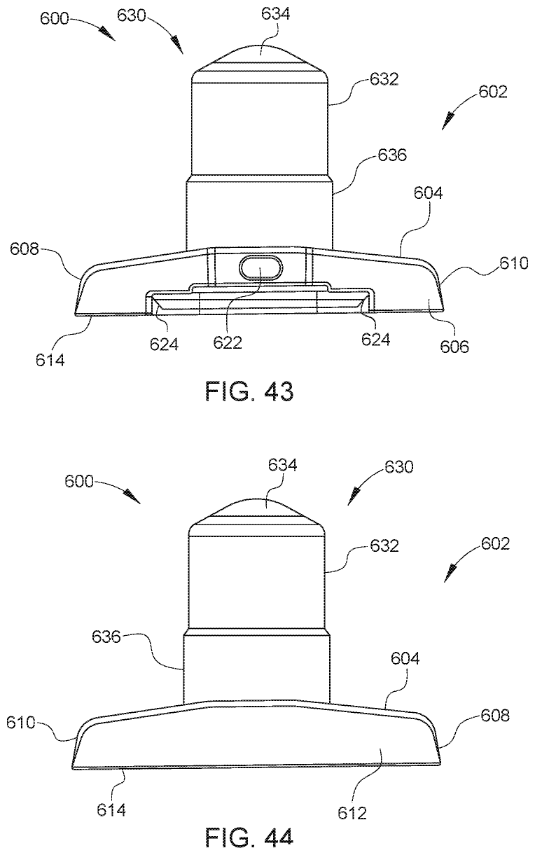

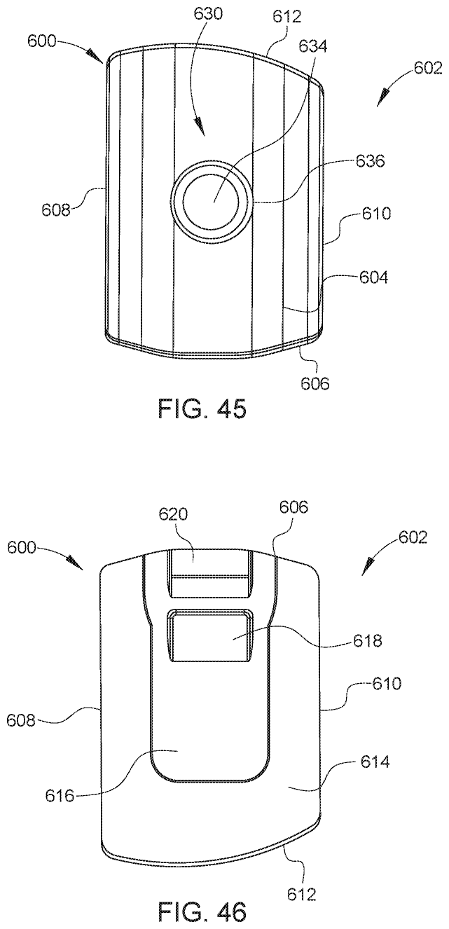

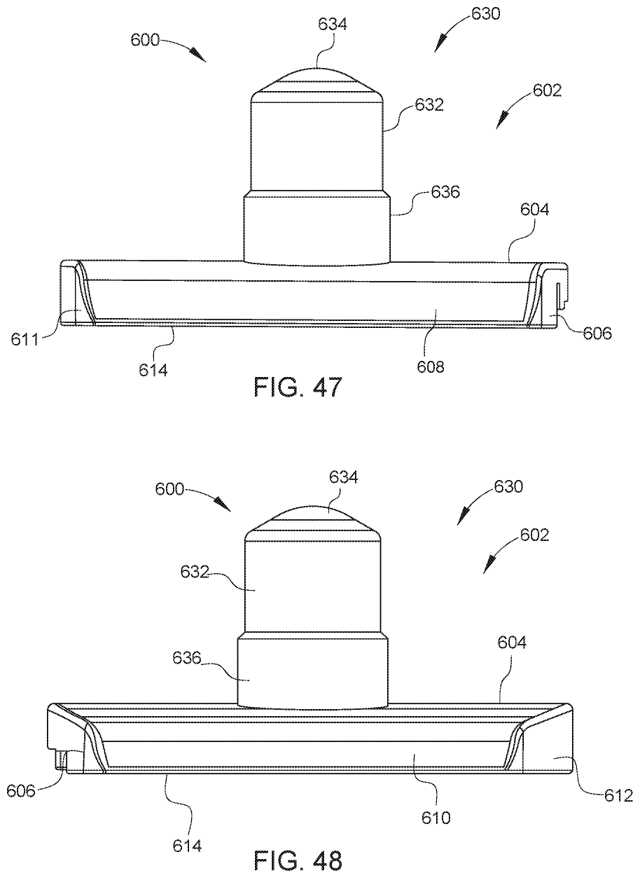

Referring now to FIGS. 41-48, an implant 600 is shown, according to an exemplary embodiment. The implant 600 may be a component of an implant system, for example an ankle arthroplasty or TAR system and as such may include one or more features configured to interface, couple, and/or engage with one or more other components of the system. The implant 600 may also be offered in various sizes and/or dimensions, for example to accommodate various sizes of patient anatomy and/or deformities or conditions with which patients may present. Furthermore, the implant 600 may be offered in configurations where one or more of the components of the implant are variously sized. For example, in some aspects the implant may be offered in configurations with a specific component in small, medium, or large sizes.

The implant 600 includes a body 602, according to the exemplary embodiment of FIGS. 41-48. The body 602 is shown to include a top surface 606 substantially opposite the body 602 from a bottom surface 614, a front end 606 substantially opposite the body 602 from a rear end 612, and a lateral side 608 substantially opposite the body 602 from a medial side 610. As shown, the implant 600 is configured for a right ankle of a patient, where the lateral and medial sides 608, 610 are identified accordingly (and thus would be opposite if the implant 600 were configured for a left ankle of a patient). As shown, the top and bottom surfaces 604, 614 are positioned in planes substantially parallel to one another. Similarly, the lateral and medial sides 608, 610 include respective surfaces at least a portion of which are positioned in planes substantially parallel to one another and substantially orthogonal to those of the top and bottom surfaces 604, 614. The front end 606 is shown to include a surface at least a portion of which is positioned in a plane perpendicular to both the planes of the lateral and medial sides 608, 610 and the top and bottom surfaces 604, 614. The rear end 612 is shown to include a curvature (e.g., a radiused portion) along at least a portion thereof, wherein at least a portion of the curved surface thereof is positioned in one or more planes substantially perpendicular to those of the top and bottom surfaces 604, 614.

The body 602 and the top surface 604 thereof are shown to include an extension 630 protruding upward (e.g., substantially perpendicular to at least a portion of the top surface 604) from the top surface 604, at least a portion of which includes a substantially cylindrical geometry. As shown in FIGS. 41-48, the extension 630 is positioned centrally relative to the top surface 604 of the body 602. However, in some aspects the extension 604 may be positioned variously at other locations about the top surface 604 of the implant 600. Further, in some aspects the implant 600 may include one or more extensions 630 positioned variously about the top surface 604 of the implant 600. For example, multiple extensions 630 may be positioned variously about the top surface 604 of the implant 600 so as to accommodate the anatomy of a specific patient (e.g., positioned to fill a distal tibial void or volume created by a cyst or other anatomic condition) and/or positioned to provide additional tibial stability in the distal tibia of a patient upon implantation.

The extension 630 as shown in FIGS. 41-48 is integral with the body 602 and the top surface 604 of the implant 600. However, in some aspects the extension 630 may be releasably couplable or otherwise engageable with the top surface 604 and/or body 602 of the implant 600. For example, a system may be provided to a physician which includes multiple implant bodies 602 and multiple extensions 630, where each of the multiple bodies 602 and extensions 630 are of a different size. Further to the previous example, each of the bodies 602 and extensions 630 may be compatible with one another such that a physician may assemble a desired implant including a desired size body 602 and extension 630 intraoperatively, where the extension 630 is couplable or otherwise engageable with the body 602. The extension 630 is shown to include a base 636 which is integral with the body 602 and the extension 630 and includes a geometry that is the same as or similar to that of the extension 630 (for example, a substantially cylindrical shape). As shown, the base 636 includes a greater lateral dimension (as shown, radius/diameter and thus circumference) than that of the extension 630. The base 636 is shown to have a hollow central portion which is occupied by at least a portion of the extension 630, with the base 636 having a height which extends at least a portion of the way up the height of the extension 630. The extension 630 and an outer surface thereof may form a substantially right angle with an upper surface of the base 636, may form a substantially oblique angle with the top surface of the base 636, or include one or more radiused or otherwise curved areas between the top surface of the base 636 and the outer surface of the extension 630. In some aspects, the base 636 may vary in size (e.g., height, diameter/radius, circumference, etc. according to the sizing of respective components of the implant 600. For example, the base 636 may have a greater lateral dimension on a smaller sized implant 600 than a larger size implant 600. Further, the base 636 may be customizable by a physician for a specific size of implant based on the specific anatomy of a patient. The extension 630 may be similar in geometry to that of the extension 230 of FIGS. 9-16 and/or 630 of FIGS. 25-32, but may also have a lesser height (e.g., a 12 mm height vs. 15 mm height on the extension 230) so as to extend further proximally into the distal tibia upon implantation. In some aspects, the implant 600 may include multiple extension augments, one or more of which may be coupled with the extension 630 and/or other augments so as to adjust (e.g., increase a height of) the extension 630 and dimensions thereof.

The extension 630 includes a shaft 632 which subsequently includes a substantially cylindrical body (e.g., with circular cross-sections and a constant radius/diameter) along at least a portion of the length/height thereof. In some aspects, the extension 630 and/or shaft 632 may include alternate bodies, for example triangular, rectangular, or other geometric prism-shaped geometries. Further, in some aspects the extension 630 and/or shaft 632 may include one or more features disposed on/in the outer surface and/or along a length thereof. For example, the extension 630 and/or shaft 632 may include one or more protrusions, depressions, protuberances, cavities, barbs, or other geometric elements either extending outward or inward from the outer surface thereof. The extension 630 is shown to include a top portion 634 which, as shown, is integral with the shaft 632 but may also be couplable/engageable in some aspects. The top portion 634 is shown to have a substantially semi-spherical or convex geometry including a rounded surface extending in a direction (and including an apex) opposite that of the body 602 relative to the shaft 632. In some aspects, the top portion 634 may include alternate geometries, for example various curvatures/radii of a convex/rounded surfaces, one or more protrusions/barbs/spikes, or other geometric components extending outward or inward from an outer surface thereof.

The bottom surface 614 of the body 602 is shown to include one or more features configured to facilitate releasable coupling and/or engagement with one or more other components of an ankle arthroplasty system (for example, an intermediate component such as a polymeric component including but not limited to those incorporated by reference herein). The bottom surface 614 includes a cavity 616 extending into the body 602 from the bottom surface 614 toward the top surface 604. In some aspects, the cavity 616 may be defined by a continuous, substantially u-shaped perimeter, with at least a portion of the perimeter including an engagement feature 624 (shown in exemplary aspects herein as a female dovetail feature and referred to hereinafter as dovetail 624) configured to facilitate bottom the aforementioned releasable coupling/engagement with one or more other components of an ankle arthroplasty system. The cavity 616 may be positioned such that the cavity 616 is in fluid communication with the front end 606 (e.g., extends into the surface 614/the body 602 from the front end 606 and/or a surface thereof, toward the rear end 612). The bottom surface 614 is further shown to include a cavity 618 and a cavity 620, which may be positioned within the cavity 616 (e.g., extend into the body 602 toward the top surface 606 from within the cavity 616). In some aspects, the cavities 618, 620 may include a substantially rectangular geometry, but may also include other geometries/volumes in alternative aspects. As shown, the cavity 620 is in fluid communication with the front end 606 and is spaced equidistant from opposing sides of the perimeter of the cavity 616. In some aspects, the cavities 616, 618, 620 may be configured such that an intermediate component of an ankle arthroplasty system may be slidably coupled with the implant 600, with the intermediate component positioned adjacent the front end 606 and slid, with an engagement portion (e.g., a male dovetail) aligned with the dovetail 624, toward the rear side 612. Such an intermediate component may also include one or more features configured to releasable couple/engage with one or more of the cavities 618, 620.

The front end 606 of the implant 600 is shown to include a recess 622 disposed on a surface thereof and extending into the body 602 toward the rear side 612. As shown, the recess 622 includes a substantially oval-shaped cross-section, but in some aspects may include other rounded geometric cross-sections or alternate geometric cross-sections. The recess 622 may be configured to engage with (e.g., receive at least a portion thereof within) one or more instruments of an ankle arthroplasty system to facilitate implantation, manipulation, or other various position/repositioning adjustments of the implant 600.

In some aspects, at least a portion of the body 602 and/or the extension 630 may include a textured surface, for example a roughness, configured to facilitate retention of the implant 600 within a distal tibia of a patient. Such a textured surface may be disposed on at least a portion of the body 602 (e.g., one or more of the top surface 604, the lateral side 608, the medial side 610, and the base 636) and/or the extension 630 in one or more patterns, according to some aspects. Further, in some aspects the body 602 and/or the extension 630 may include one or more ingrowth surfaces configured to accommodate bony ingrowth and/or packing by a physician with a biologic material.

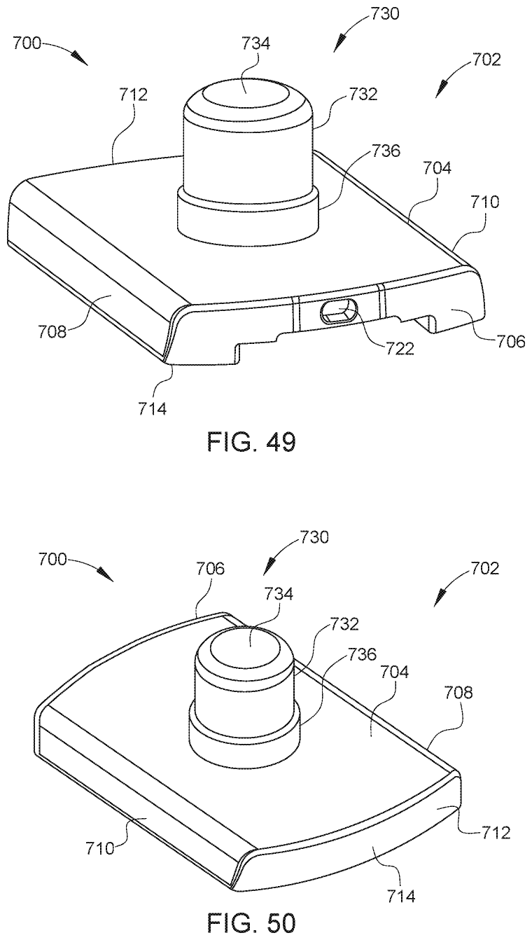

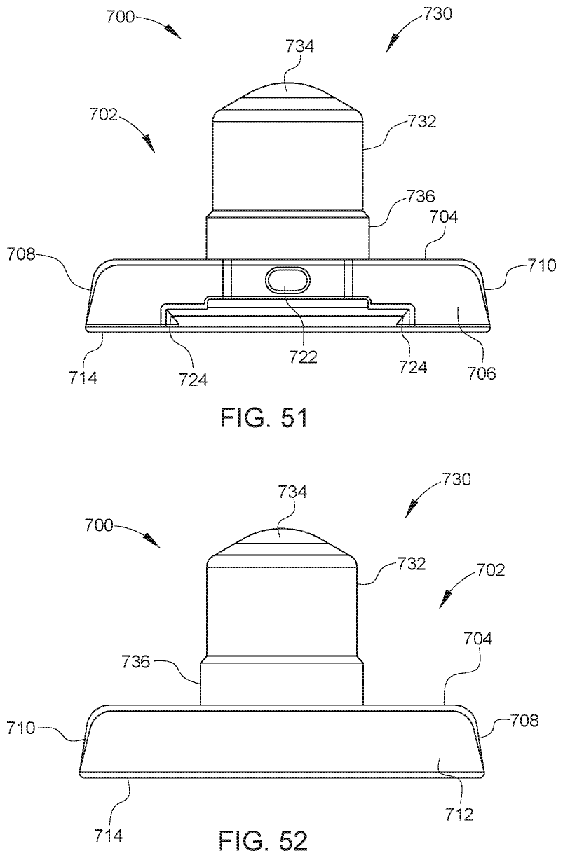

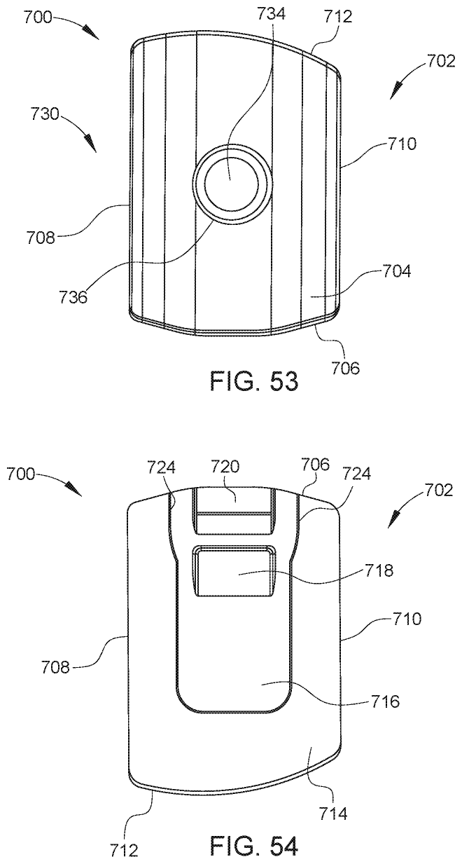

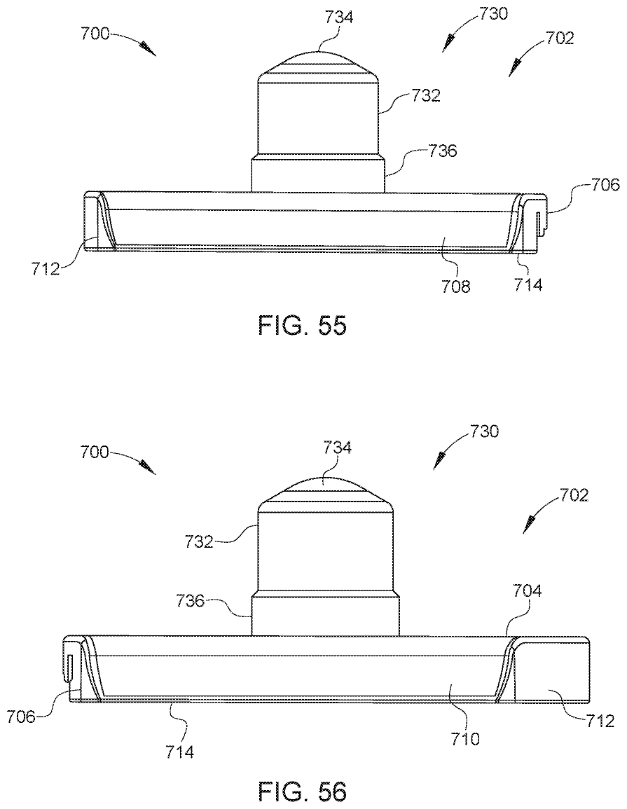

Referring now to FIGS. 49-56, an implant 700 is shown, according to an exemplary embodiment. The implant 700 may be a component of an implant system, for example an ankle arthroplasty or TAR system and as such may include one or more features configured to interface, couple, and/or engage with one or more other components of the system. The implant 700 may also be offered in various sizes and/or dimensions, for example to accommodate various sizes of patient anatomy and/or deformities or conditions with which patients may present. Furthermore, the implant 700 may be offered in configurations where one or more of the components of the implant 700 are variously sized. For example, in some aspects the implant 700 may be offered in configurations with a specific component in small, medium, or large sizes.

The implant 700 includes a body 702, according to the exemplary embodiment of FIGS. 49-56. The body 702 is shown to include a top surface 706 substantially opposite the body 702 from a bottom surface 714, a front end 706 substantially opposite the body 702 from a rear end 712, and a lateral side 708 substantially opposite the body 702 from a medial side 710. As shown, the implant 700 is configured for a right ankle of a patient, where the lateral and medial sides 708, 710 are identified accordingly (and thus would be opposite if the implant 700 were configured for a left ankle of a patient). As shown, the top and bottom surfaces 704, 714 are positioned in planes substantially parallel to one another. Similarly, the lateral and medial sides 708, 710 include respective surfaces at least a portion of which are positioned in planes substantially parallel to one another and substantially orthogonal to those of the top and bottom surfaces 704, 714. The front end 706 is shown to include a surface at least a portion of which is positioned in a plane perpendicular to both the planes of the lateral and medial sides 708, 710 and the top and bottom surfaces 704, 714. The rear end 712 is shown to include a curvature (e.g., a radiused portion) along at least a portion thereof, wherein at least a portion of the curved surface thereof is positioned in one or more planes substantially perpendicular to those of the top and bottom surfaces 704, 714.

The body 702 and the top surface 704 thereof are shown to include an extension 730 protruding upward (e.g., substantially perpendicular to at least a portion of the top surface 704) from the top surface 704, at least a portion of which includes a substantially cylindrical geometry. As shown in FIGS. 49-56, the extension 730 is positioned centrally relative to the top surface 704 of the body 702. However, in some aspects the extension 704 may be positioned variously at other locations about the top surface 704 of the implant 700. Further, in some aspects the implant 700 may include one or more extensions 730 positioned variously about the top surface 704 of the implant 700. For example, multiple extensions 730 may be positioned variously about the top surface 704 of the implant 700 so as to accommodate the anatomy of a specific patient (e.g., positioned to fill a distal tibial void or volume created by a cyst or other anatomic condition) and/or positioned to provide additional tibial stability in the distal tibia of a patient upon implantation.

The extension 730 as shown in FIGS. 49-56 is integral with the body 702 and the top surface 704 of the implant 700. However, in some aspects the extension 730 may be releasably couplable or otherwise engageable with the top surface 704 and/or body 702 of the implant 700. For example, a system may be provided to a physician which includes multiple implant bodies 702 and multiple extensions 730, where each of the multiple bodies 702 and extensions 730 are of a different size. Further to the previous example, each of the bodies 702 and extensions 730 may be compatible with one another such that a physician may assemble a desired implant 700 including a desired size body 702 and extension 730 intraoperatively, where the extension 730 is couplable or otherwise engageable with the body 702. The extension 730 is shown to include a base 736 which is integral with the body 702 and the extension 730 and includes a geometry that is the same as or similar to that of the extension 730 (for example, a substantially cylindrical shape). As shown, the base 736 includes a greater lateral dimension (as shown, radius/diameter and thus circumference) than that of the extension 730. The base 736 is shown to have a hollow central portion which is occupied by at least a portion of the extension 730, with the base 736 having a height which extends at least a portion of the way up the height of the extension 730. The extension 730 and an outer surface thereof may form a substantially right angle with an upper surface of the base 736, may form a substantially oblique angle with the top surface of the base 736, or include one or more radiused or otherwise curved areas between the top surface of the base 736 and the outer surface of the extension 730. In some aspects, the base 736 may vary in size (e.g., height, diameter/radius, circumference, etc. according to the sizing of respective components of the implant 700. For example, the base 736 may have a greater lateral dimension on a smaller sized implant 700 than a larger size implant 700. Further, the base 736 may be customizable by a physician for a specific size of implant 700 based on the specific anatomy of a patient. The extension 730 may be similar in geometry to that of the extension 230 of FIGS. 9-16, 430 of FIGS. 25-32, and/or 630 of FIGS. 41-48 but may also have a different height/width/other dimensions so as to extend further proximally into the distal tibia upon implantation. In some aspects, the implant 700 may include multiple extension augments, one or more of which may be coupled with the extension 730 and/or other augments so as to adjust (e.g., increase a height of) the extension 730 and dimensions thereof.

The extension 730 includes a shaft 732 which subsequently includes a substantially cylindrical body (e.g., with circular cross-sections and a constant radius/diameter) along at least a portion of the length/height thereof. In some aspects, the extension 730 and/or shaft 732 may include alternate bodies, for example triangular, rectangular, or other geometric prism-shaped geometries. Further, in some aspects the extension 730 and/or shaft 732 may include one or more features disposed on/in the outer surface and/or along a length thereof. For example, the extension 730 and/or shaft 732 may include one or more protrusions, depressions, protuberances, cavities, barbs, or other geometric elements either extending outward or inward from the outer surface thereof. The extension 730 is shown to include a top portion 734 which, as shown, is integral with the shaft 732 but may also be couplable/engageable in some aspects. The top portion 734 is shown to have a substantially semi-spherical or convex geometry including a rounded surface extending in a direction (and including an apex) opposite that of the body 702 relative to the shaft 732. In some aspects, the top portion 734 may include alternate geometries, for example various curvatures/radii of a convex/rounded surfaces, one or more protrusions/barbs/spikes, or other geometric components extending outward or inward from an outer surface thereof.

The bottom surface 714 of the body 702 is shown to include one or more features configured to facilitate releasable coupling and/or engagement with one or more other components of an ankle arthroplasty system (for example, an intermediate component such as a polymeric component including but not limited to those incorporated by reference herein). The bottom surface 714 includes a cavity 716 extending into the body 702 from the bottom surface 714 toward the top surface 704. In some aspects, the cavity 716 may be defined by a continuous, substantially u-shaped perimeter, with at least a portion of the perimeter including an engagement feature 724 (shown in exemplary aspects herein as a female dovetail feature and referred to hereinafter as dovetail 724) configured to facilitate bottom the aforementioned releasable coupling/engagement with one or more other components of an ankle arthroplasty system. The cavity 716 may be positioned such that the cavity 716 is in fluid communication with the front end 706 (e.g., extends into the surface 714/the body 702 from the front end 706 and/or a surface thereof, toward the rear end 712). The bottom surface 714 is further shown to include a cavity 718 and a cavity 720, which may be positioned within the cavity 716 (e.g., extend into the body 702 toward the top surface 706 from within the cavity 716). In some aspects, the cavities 718, 720 may include a substantially rectangular geometry, but may also include other geometries/volumes in alternative aspects. As shown, the cavity 720 is in fluid communication with the front end 706 and is spaced equidistant from opposing sides of the perimeter of the cavity 716. In some aspects, the cavities 716, 718, 720 may be configured such that an intermediate component of an ankle arthroplasty system may be slidably coupled with the implant 700, with the intermediate component positioned adjacent the front end 706 and slid, with an engagement portion (e.g., a male dovetail) aligned with the dovetail 724, toward the rear side 712. Such an intermediate component may also include one or more features configured to releasable couple/engage with one or more of the cavities 718, 720.

The front end 706 of the implant 700 is shown to include a recess 722 disposed on a surface thereof and extending into the body 702 toward the rear side 712. As shown, the recess 722 includes a substantially oval-shaped cross-section, but in some aspects may include other rounded geometric cross-sections or alternate geometric cross-sections. The recess 722 may be configured to engage with (e.g., receive at least a portion thereof within) one or more instruments of an ankle arthroplasty system to facilitate implantation, manipulation, or other various position/repositioning adjustments of the implant 700.

In some aspects, at least a portion of the body 702 and/or the extension 730 may include a textured surface, for example a roughness, configured to facilitate retention of the implant 700 within a distal tibia of a patient. Such a textured surface may be disposed on at least a portion of the body 702 (e.g., one or more of the top surface 704, the lateral side 708, the medial side 710, and the base 736) and/or the extension 730 in one or more patterns, according to some aspects. Further, in some aspects the body 702 and/or the extension 730 may include one or more ingrowth surfaces configured to accommodate bony ingrowth and/or packing by a physician with a biologic material.

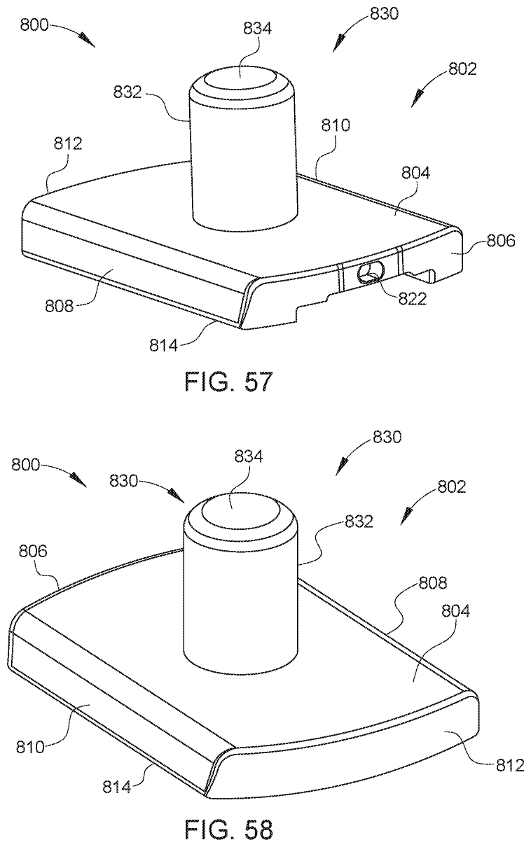

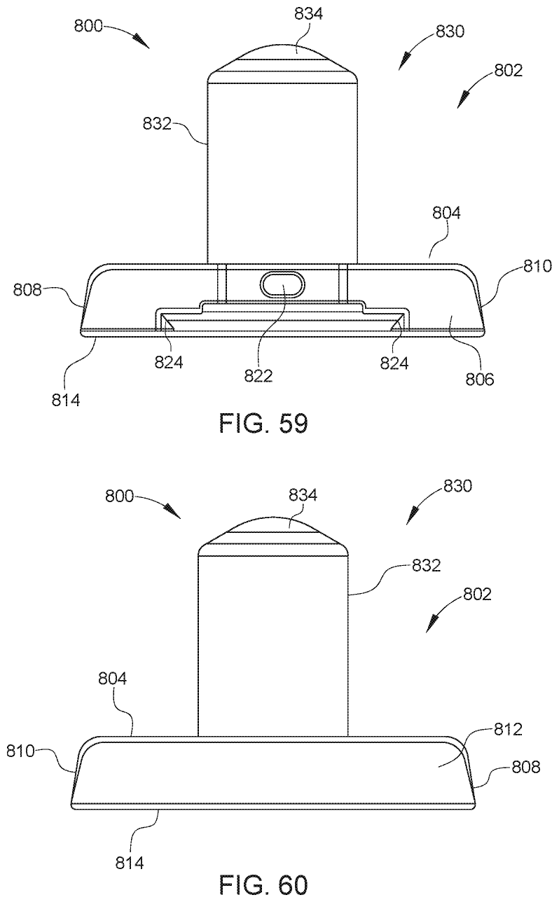

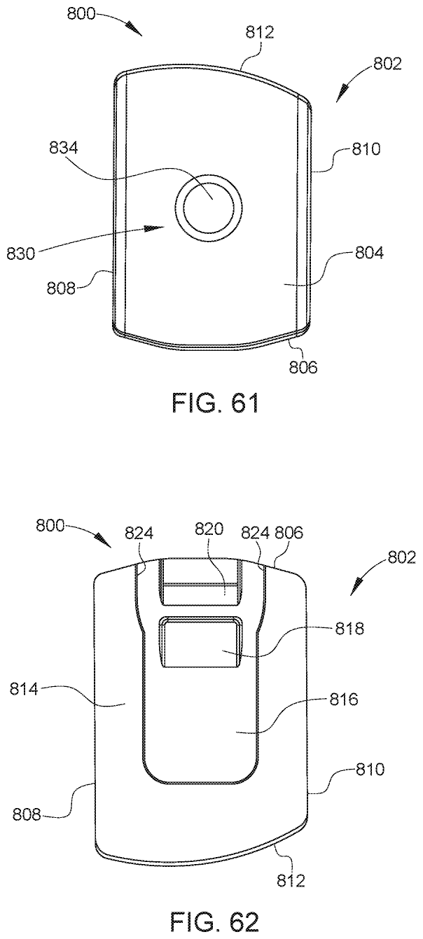

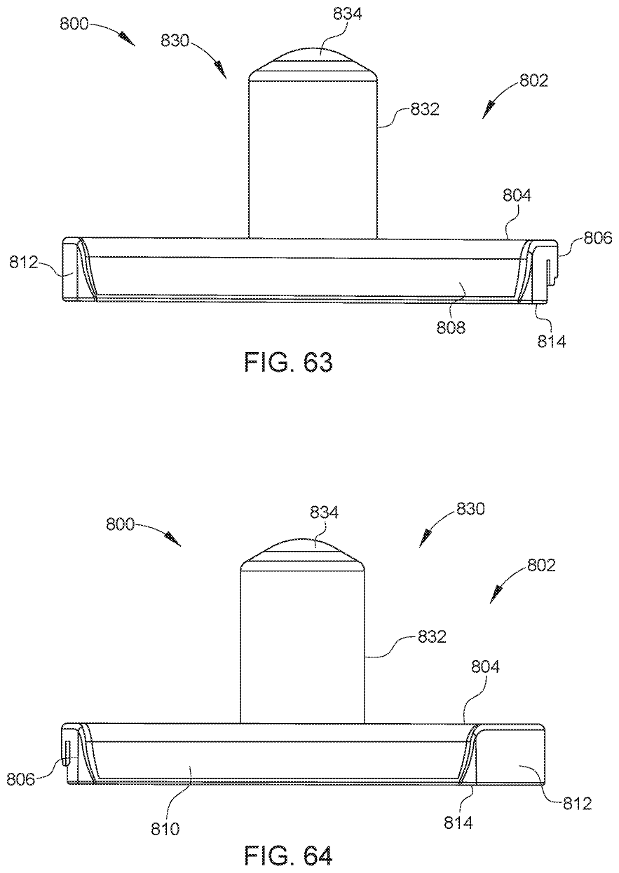

Referring now to FIGS. 57-64, an implant 800 is shown, according to an exemplary embodiment. The implant 800 may be a component of an implant system, for example an ankle arthroplasty or TAR system and as such may include one or more features configured to interface, couple, and/or engage with one or more other components of the system. The implant 800 may also be offered in various sizes and/or dimensions, for example to accommodate various sizes of patient anatomy and/or deformities or conditions with which patients may present. Furthermore, the implant 800 may be offered in configurations where one or more of the components of the implant 800 are variously sized. For example, in some aspects the implant 800 may be offered in configurations with a specific component in small, medium, or large sizes.

The implant 800 includes a body 802, according to the exemplary embodiment of FIGS. 57-64. The body 802 is shown to include a top surface 806 substantially opposite the body 802 from a bottom surface 814, a front end 806 substantially opposite the body 802 from a rear end 812, and a lateral side 808 substantially opposite the body 802 from a medial side 810. As shown, the implant 800 is configured for a right ankle of a patient, where the lateral and medial sides 808, 810 are identified accordingly (and thus would be opposite if the implant 800 were configured for a left ankle of a patient). As shown, the top and bottom surfaces 804, 814 are positioned in planes substantially parallel to one another. Similarly, the lateral and medial sides 808, 810 include respective surfaces at least a portion of which are positioned in planes substantially parallel to one another and substantially orthogonal to those of the top and bottom surfaces 804, 814. The front end 806 is shown to include a surface at least a portion of which is positioned in a plane perpendicular to both the planes of the lateral and medial sides 808, 810 and the top and bottom surfaces 804, 814. The rear end 812 is shown to include a curvature (e.g., a radiused portion) along at least a portion thereof, wherein at least a portion of the curved surface thereof is positioned in one or more planes substantially perpendicular to those of the top and bottom surfaces 804, 814.

The body 802 and the top surface 804 thereof are shown to include an extension 830 protruding upward (e.g., substantially perpendicular to at least a portion of the top surface 804) from the top surface 804, at least a portion of which includes a substantially cylindrical geometry. As shown in FIGS. 57-64, the extension 830 is positioned centrally relative to the top surface 804 of the body 802. However, in some aspects the extension 804 may be positioned variously at other locations about the top surface 804 of the implant 800. Further, in some aspects the implant 800 may include one or more extensions 830 positioned variously about the top surface 804 of the implant 800. For example, multiple extension 830 may be positioned variously about the top surface 804 of the implant 800 so as to accommodate the anatomy of a specific patient (e.g., positioned to fill a distal tibial void or volume created by a cyst or other anatomic condition) and/or positioned to provide additional tibial stability in the distal tibia of a patient upon implantation.

The extension 830 as shown in FIGS. 57-64 is integral with the body 802 and the top surface 804 of the implant 800. However, in some aspects the extension 830 may be releasably couplable or otherwise engageable with the top surface 804 and/or body 802 of the implant 800. For example, a system may be provided to a physician which includes multiple implant bodies 802 and multiple extensions 830, where each of the multiple bodies 802 and extensions 830 are of a different size. Further to the previous example, each of the bodies 802 and extensions 830 may be compatible with one another such that a physician may assemble a desired implant 800 including a desired size body 802 and extension 830 intraoperatively, where the extension 830 is couplable or otherwise engageable with the body 802. The extension 830 may be similar in geometry to that of the extension 130 of FIGS. 1-8, 330 of FIGS. 17-24, and/or 730 of FIGS. 41-48 but may also have a different height or other various dimensions (e.g., so as to extend further proximally into the distal tibia upon implantation). In some aspects, the implant 800 may include multiple extension augments, one or more of which may be coupled with the extension 830 and/or other augments so as to adjust (e.g., increase a height of) the extension 830 and dimensions thereof.

The extension 830 includes a shaft 832 which subsequently includes a substantially cylindrical body (e.g., with circular cross-sections and a constant radius/diameter) along at least a portion of the length/height thereof. In some aspects, the extension 830 and/or shaft 832 may include alternate bodies, for example triangular, rectangular, or other geometric prism-shaped geometries. Further, in some aspects the extension 830 and/or shaft 832 may include one or more features disposed on/in the outer surface and/or along a length thereof. For example, the extension 830 and/or shaft 832 may include one or more protrusions, depressions, protuberances, cavities, barbs, or other geometric elements either extending outward or inward from the outer surface thereof. The extension 830 is shown to include a top portion 834 which, as shown, is integral with the shaft 832 but may also be couplable/engageable in some aspects. The top portion 834 is shown to have a substantially semi-spherical or convex geometry including a rounded surface extending in a direction (and including an apex) opposite that of the body 802 relative to the shaft 832. In some aspects, the top portion 834 may include alternate geometries, for example various curvatures/radii of a convex/rounded surfaces, one or more protrusions/barbs/spikes, or other geometric components extending outward or inward from an outer surface thereof.

The bottom surface 814 of the body 802 is shown to include one or more features configured to facilitate releasable coupling and/or engagement with one or more other components of an ankle arthroplasty system (for example, an intermediate component such as a polymeric component including but not limited to those incorporated by reference herein). The bottom surface 814 includes a cavity 816 extending into the body 802 from the bottom surface 814 toward the top surface 804. In some aspects, the cavity 816 may be defined by a continuous, substantially u-shaped perimeter, with at least a portion of the perimeter including an engagement feature 824 (shown in exemplary aspects herein as a female dovetail feature and referred to hereinafter as dovetail 824) configured to facilitate bottom the aforementioned releasable coupling/engagement with one or more other components of an ankle arthroplasty system. The cavity 816 may be positioned such that the cavity 816 is in fluid communication with the front end 806 (e.g., extends into the surface 814/the body 802 from the front end 806 and/or a surface thereof, toward the rear end 812). The bottom surface 814 is further shown to include a cavity 818 and a cavity 820, which may be positioned within the cavity 816 (e.g., extend into the body 802 toward the top surface 806 from within the cavity 816). In some aspects, the cavities 818, 820 may include a substantially rectangular geometry, but may also include other geometries/volumes in alternative aspects. As shown, the cavity 820 is in fluid communication with the front end 806 and is spaced equidistant from opposing sides of the perimeter of the cavity 816. In some aspects, the cavities 816, 818, 820 may be configured such that an intermediate component of an ankle arthroplasty system may be slidably coupled with the implant 800, with the intermediate component positioned adjacent the front end 806 and slid, with an engagement portion (e.g., a male dovetail) aligned with the dovetail 824, toward the rear side 812. Such an intermediate component may also include one or more features configured to releasable couple/engage with one or more of the cavities 818, 820.

The front end 806 of the implant 800 is shown to include a recess 822 disposed on a surface thereof and extending into the body 802 toward the rear side 812. As shown, the recess 822 includes a substantially oval-shaped cross-section, but in some aspects may include other rounded geometric cross-sections or alternate geometric cross-sections. The recess 822 may be configured to engage with (e.g., receive at least a portion thereof within) one or more instruments of an ankle arthroplasty system to facilitate implantation, manipulation, or other various position/repositioning adjustments of the implant 800.

In some aspects, at least a portion of the body 802 and/or the extension 830 may include a textured surface, for example a roughness, configured to facilitate retention of the implant 800 within a distal tibia of a patient. Such a texture surface may be disposed on at least a portion of the body 802 (e.g., one or more of the top surface 804, the lateral side 808, the medial side 810) and/or the extension 830 in one or more patterns, according to some aspects. Further, in some aspects the body 802 and/or the extension 830 may include one or more ingrowth surfaces configured to accommodate bony ingrowth and/or packing by a physician with a biologic material.

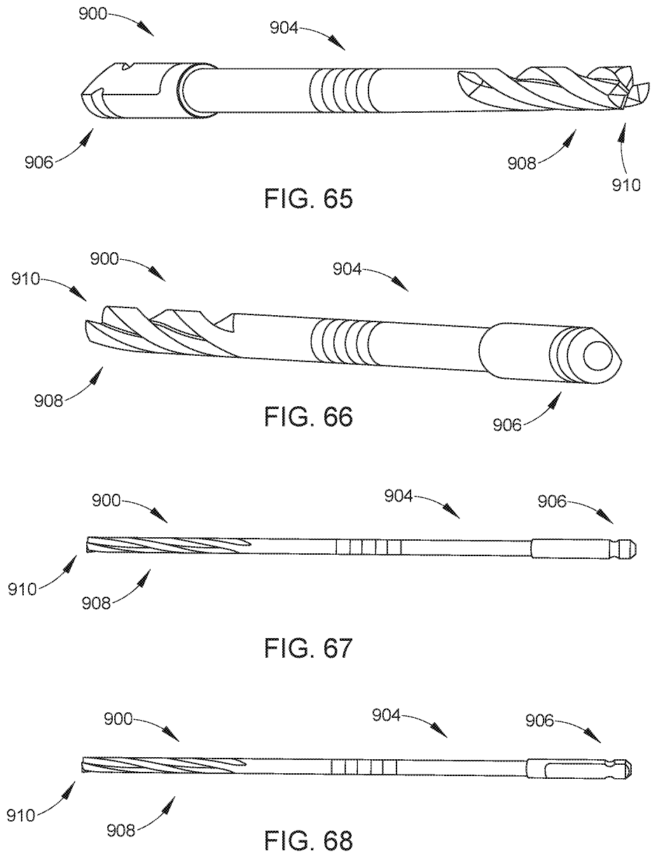

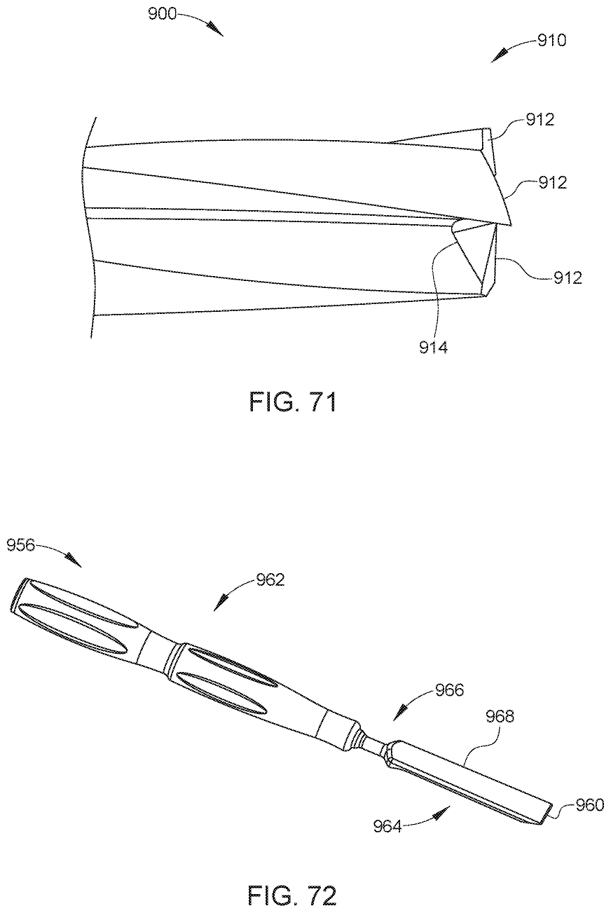

Referring now to FIGS. 65-71, an instrument 900 is shown, according to an exemplary embodiment. The instrument 900, shown and described herein as a drill bit, may be compatible with one or more other instruments common to operating rooms and/or orthopedic procedures including but not limited to arthroplasty procedures, for example total ankle arthroplasty procedures. The instrument 900 may be implemented in conjunction with a powered instrument (e.g., releasably coupled, engaged with, etc.) so as to create a void in one or more bones of a patient as part of performing an arthroplasty procedure such as an ankle arthroplasty procedure.

The instrument 900 is shown to include a shaft 904 disposed between a proximal end 906 and distal end 908 of the instrument 900. The proximal end 906 may include one or more engagement features configured to facilitate releasable coupling/engagement with powered instruments. Such engagement features may include those compatible with multiple instruments and considered common or universal connections so as to facilitate compatibility with various different appropriate powered instruments. The proximal end may include one or more flats, notches, grooves, protrusions, or other features configured to engage with complimentary features so as to facilitate releasable coupling/engagement with the aforementioned instruments and, accordingly, implementation in surgical procedures.

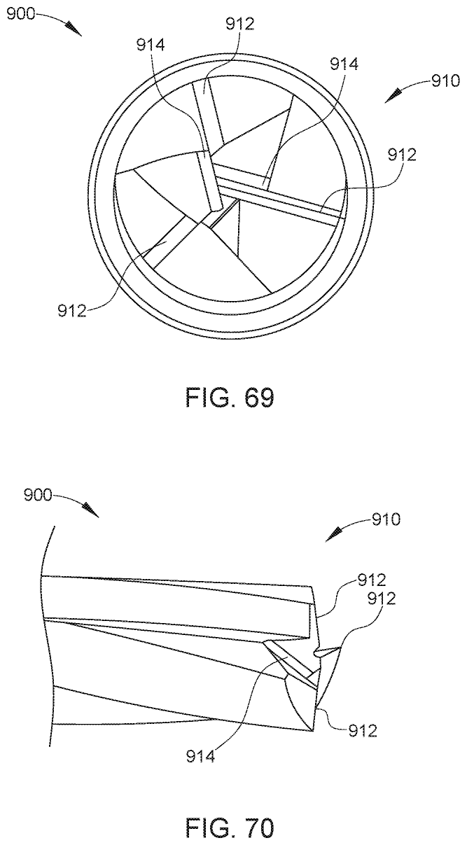

The instrument 900 is shown to include a cutting portion 910 at the distal end 908 thereof, wherein the cutting portion 910 includes one or more cutting edges and/or relief areas thereof configured to facilitate cutting and removal of bone when the instrument is implemented (in conjunction with a powered instrument) in a surgical procedure. As shown in at least FIG. 69, the cutting portion 910 includes three cutting edges 912 and three relief areas 914, with the cutting edges 912 and relief areas 914 positioned in an alternating fashion (e.g., a cutting edge 912 is positioned between two relief areas 914). Each of the cutting edges 912 is positioned at a distal-most portion of the cutting portion 910, and each of the relief areas 914 extend from the distal-most portion of the cutting portion 910 proximally toward the shaft 904. In some aspects, the cutting portion 910 may include various quantities and/or positioning of the cutting edges 912 and the relief areas 914.

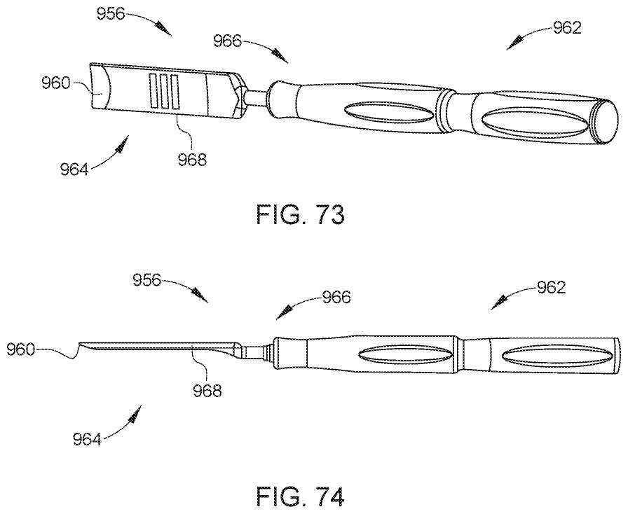

Referring now to FIGS. 72-74, an instrument 956 is shown, according to an exemplary embodiment. The instrument 956 may be implemented in various orthopedic procedures, including not limited to arthroplasty procedures such as ankle arthroplasty procedures. The instrument 956 is shown to include a handle 962 which is integral (or, in some aspects, may be coupled with) with, via a coupling 966, an end portion 964. The handle 962 may include one or more depressions, protrusions, or other geometric aspects configured to facilitate comfortable gripping of the handle 962 by a physician. The end portion 964 is shown to include a head portion 968 extending from the coupling 966. The head portion 968 is shown to include an engagement surface 960 positioned at a terminal end thereof, where the engagement surface is at least one of angled and/or curved. In some aspects, the end portion 964 may include a curvature along at least a portion thereof so as to facilitate use up against anatomic components which may also include curvatures on one or more surfaces.

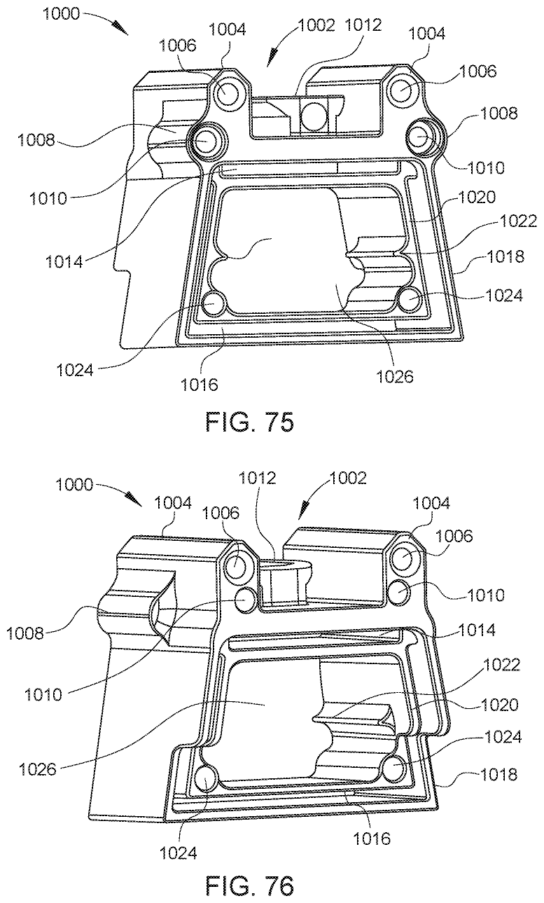

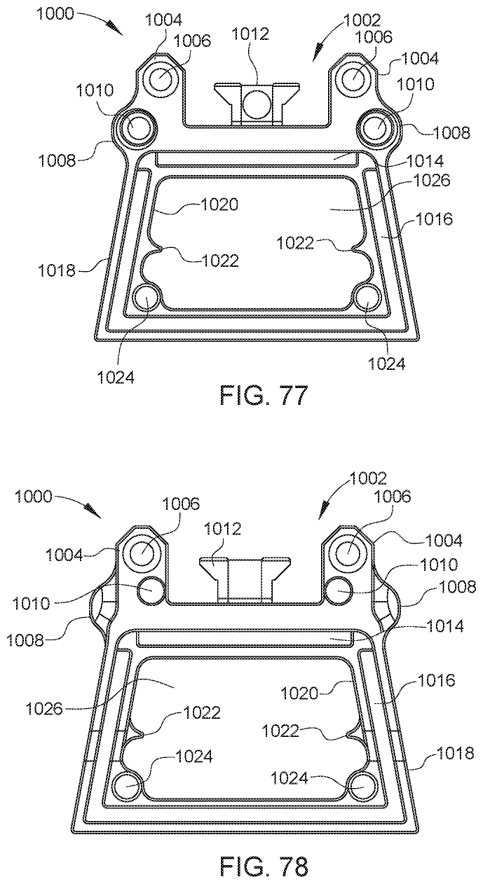

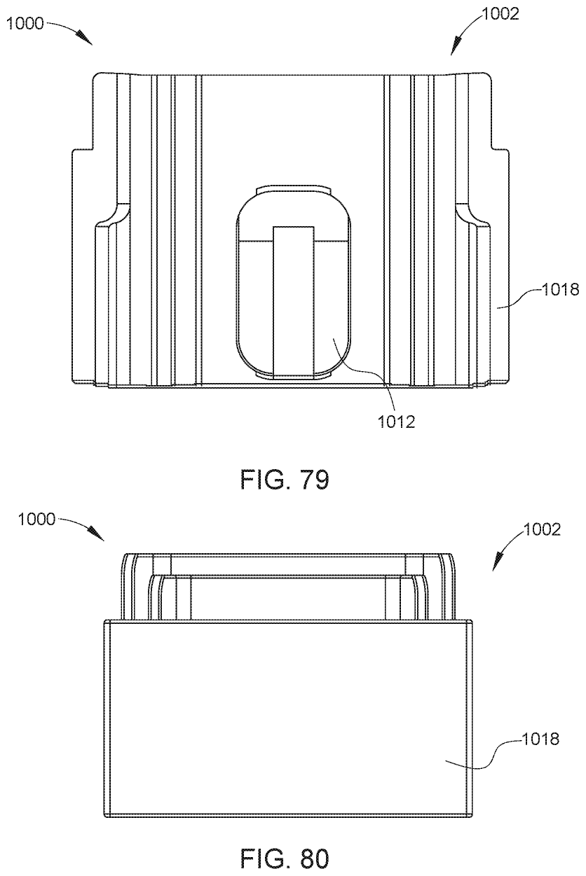

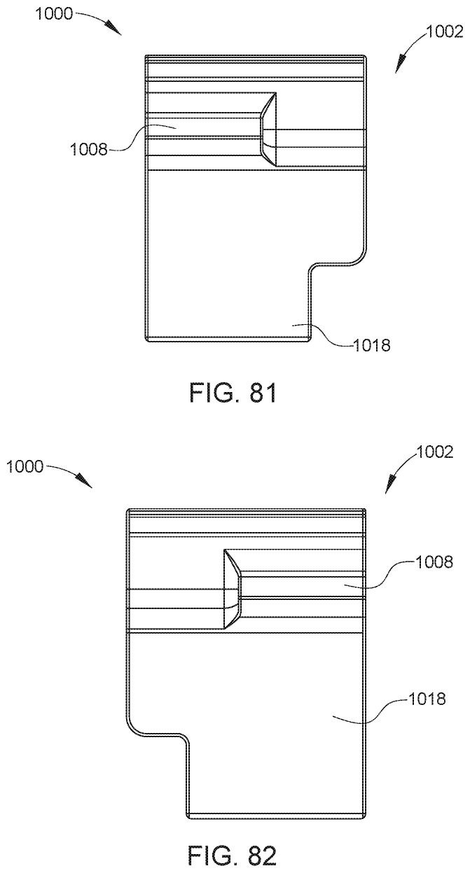

Referring now to FIGS. 75-82, an instrument 1000 is shown, according to an exemplary embodiment. The instrument 1000 may be couplable with one or more components of an ankle arthroplasty system such as alignment instruments, including but not limited to those incorporated by reference herein. In some aspects, the instrument 1000 may be releasably couplable with one or more such components, for example via one or more features of the instrument 1000. The instrument 1000 may be implemented in ankle arthroplasty procedure to guide at least one of drilling and/or cutting to the distal tibia and/or the talus of a patient, with the instrument 1000 configured to receive one or more cutting instruments at least partially therein and/or therethrough at least one opening/slot thereof.

The instrument 1000 is shown to include a body 1002, from which a pair of extension 1004 extend upward in a direction substantially parallel to one another. Each of the extensions 1004 includes at least one opening 1006 extending therethrough from a front surface to a rear surface of the body 1002. The body 1002 further includes a pair of protrusions 1008 extending laterally from the body 1002 in directions substantially opposite one another. Each of the protrusions 1008 includes an opening 1010 extending therethrough from a top surface to a bottom surface of the body 1002. The openings 1006 may include central axes which are substantially parallel to one another, whereas the openings 1010 may include central axes which converge (moving from a top/front surface of the instrument 1000 to a bottom/rear surface of the instrument 1000).

The instrument 1000 is further shown to include an engagement feature 1012 extending upward from the body 1002, according to one aspect. The engagement feature 1012, as shown in FIG. 75, is positioned substantially equidistant from and in between the pair of extensions 1004 and includes a male dovetail feature at a terminal portion thereof. In some aspects, the engagement feature 1012 may be configured to be received by a complementary feature of an alignment system so as to facilitate releasable coupling therewith (e.g., via a female dovetail component).

The body 1002 of the instrument 1000 includes a slot 1014 positioned centrally thereon and extending through the body 1002 from the top surface through to the bottom surface. As shown, the slot 1014 is substantially horizontal but in some aspects may be curved and/or angled. The body is further shown to include a slot 1016, which has a substantially u-shaped geometry, which begins/ends at points adjacent to the lateral boundaries of the slot 1014. In some aspects, the two vertical aspects of the u-shape of slot 1016 may be converging (e.g., angled toward the opening of the “U”) such that, collectively, the slot 1014 and the slot 1016 as positioned form a majority of a perimeter of a trapezoid.

The slot 1016 is defined outwardly by an outer frame 1018, which establishes a portion of the outermost surface of the instrument 1018. Similarly, the slot 1016 is defined inwardly by an inner frame 1020, which has a similar trapezoidal shape to that formed by the slots 1014, 1016. The inner frame 1020 further defines a cavity 1026 therein, which allows for visualization of bone beneath the instrument 1000 by a physician when the instrument 1000 is positioned adjacent bony anatomy. The inner frame 1020 further includes a pair of markers 1022 (e.g., protrusions) positioned on the lateral edges of the inner frame 1020 and extending into the cavity 1026. The markers 1022 may be indicative of various aspects of the anatomy, for example a joint line, to facilitate positioning of the instrument 1000. As shown, the inner frame 1020 includes a pair of markers 1022, but in some aspects may include 3 or more markers 1022. The inner frame 1020 further includes a pair of openings 1024 positioned adjacent corners of the trapezoidal shape of the inner frame 1020 and extending from a top surface of the body 1002 through to a bottom surface thereof. In some aspects, central axes of the openings 1024 may be substantially parallel, while in other aspects the axes may be converging or diverging.

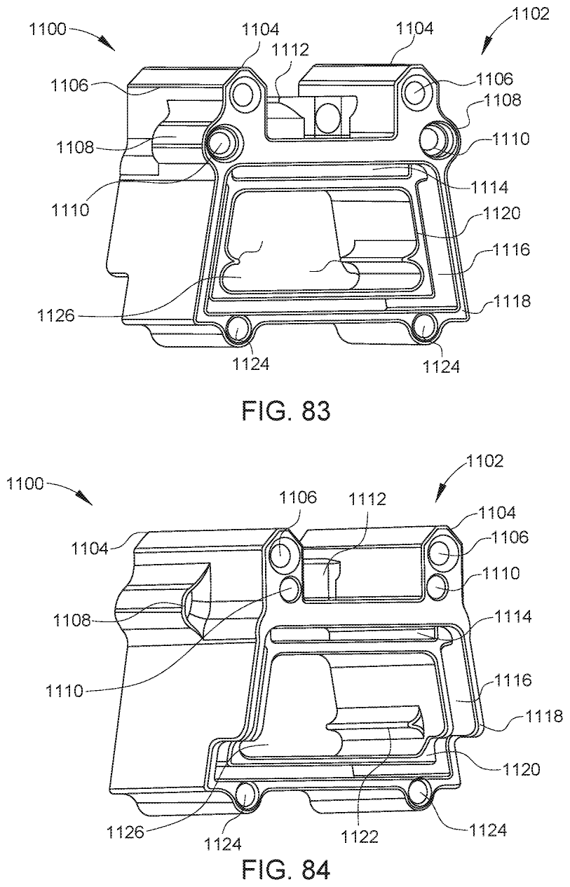

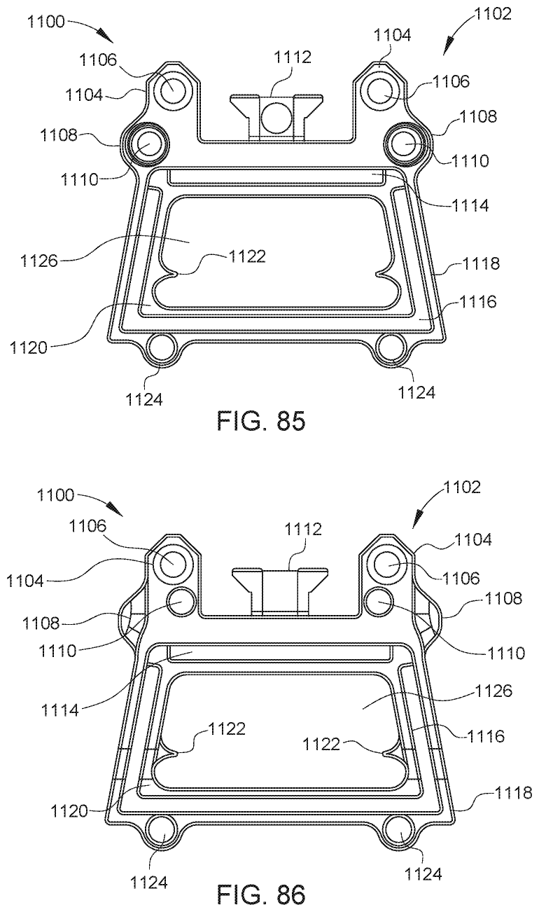

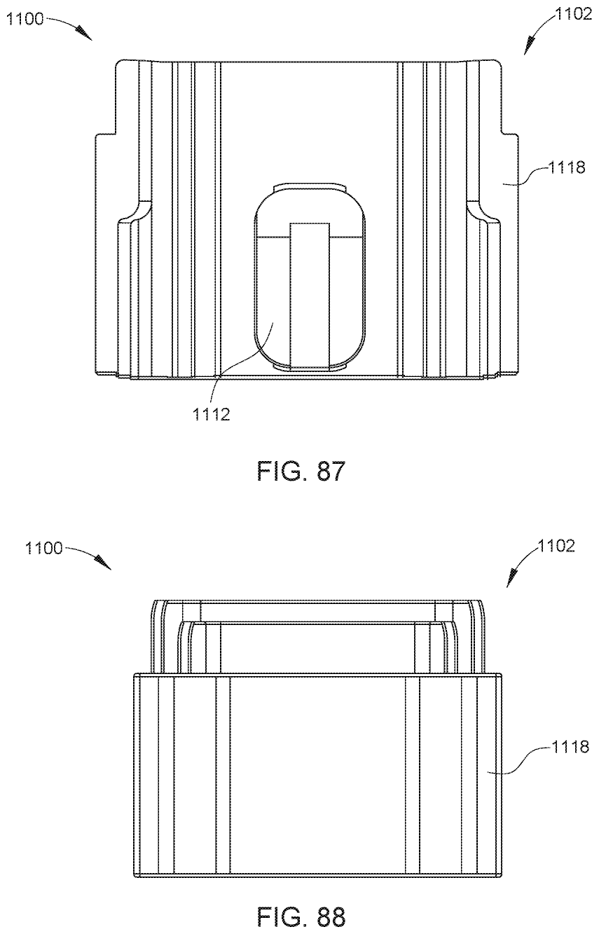

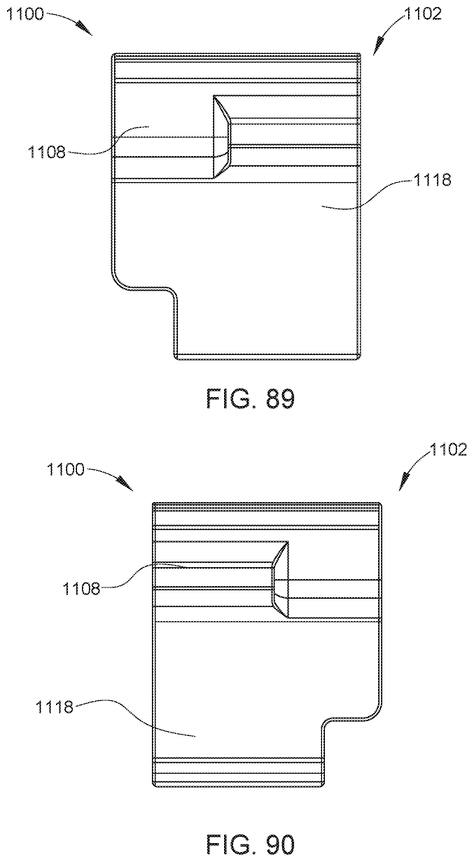

Referring now to FIGS. 83-90, an instrument 1100 is shown, according to an exemplary embodiment. The instrument 1100 may be couplable with one or more components of an ankle arthroplasty system such as alignment instruments, including but not limited to those incorporated by reference herein. In some aspects, the instrument 1100 may be releasably couplable with one or more such components, for example via one or more features of the instrument 1100. The instrument 1100 may be implemented in ankle arthroplasty procedure to guide at least one of drilling and/or cutting to the distal tibia and/or the talus of a patient, with the instrument 1100 configured to receive one or more cutting instruments at least partially therein and/or therethrough at least one opening/slot thereof.

The instrument 1100 is shown to include a body 1102, from which a pair of extension 1104 extend upward in a direction substantially parallel to one another. Each of the extensions 1104 includes at least one opening 1106 extending therethrough from a front surface to a rear surface of the body 1102. The body 1102 further includes a pair of protrusions 1108 extending laterally from the body 1102 in directions substantially opposite one another. Each of the protrusions 1108 includes an opening 1110 extending therethrough from a top surface to a bottom surface of the body 1102. The openings 1106 may include central axes which are substantially parallel to one another, whereas the openings 1110 may include central axes which converge (moving from a top/front surface of the instrument 1100 to a bottom/rear surface of the instrument 1100).

The instrument 1100 is further shown to include an engagement feature 1112 extending upward from the body 1102, according to one aspect. The engagement feature 1112, as shown in FIG. 83, is positioned substantially equidistant from and in between the pair of extensions 1104 and includes a male dovetail feature at a terminal portion thereof. In some aspects, the engagement feature 1112 may be configured to be received by a complementary feature of an alignment system so as to facilitate releasable coupling therewith (e.g., via a female dovetail component).

The body 1102 of the instrument 1100 includes a slot 1114 positioned centrally thereon and extending through the body 1102 from the top surface through to the bottom surface. As shown, the slot 1114 is substantially horizontal but in some aspects may be curved and/or angled. The body is further shown to include a slot 1116, which has a substantially u-shaped geometry, which begins/ends at points adjacent to the lateral boundaries of the slot 1114. In some aspects, the two vertical aspects of the u-shape of slot 1116 may be converging (e.g., angled toward the opening of the “U”) such that, collectively, the slot 1114 and the slot 1116 as positioned form a majority of a perimeter of a trapezoid.

The slot 1116 is defined outwardly by an outer frame 1118, which establishes a portion of the outermost surface of the instrument 1118. Similarly, the slot 1116 is defined inwardly by an inner frame 1120, which has a similar trapezoidal shape to that formed by the slots 1114, 1116. The inner frame 1120 further defines a cavity 1126 therein, which allows for visualization of bone beneath the instrument 1100 by a physician when the instrument 1100 is positioned adjacent bony anatomy. The inner frame 1120 further includes a pair of markers 1122 (e.g., protrusions) positioned on the lateral edges of the inner frame 1120 and extending into the cavity 1126. The markers 1122 may be indicative of various aspects of the anatomy, for example a joint line, to facilitate positioning of the instrument 1100. As shown, the inner frame 1120 includes a pair of markers 1122, but in some aspects may include 3 or more markers 1122. The inner frame 1120 further includes a pair of openings 1124 positioned adjacent corners of the trapezoidal shape of the inner frame 1120 and extending from a top surface of the body 1102 through to a bottom surface thereof. In some aspects, central axes of the openings 1124 may be substantially parallel, while in other aspects the axes may be converging or diverging.

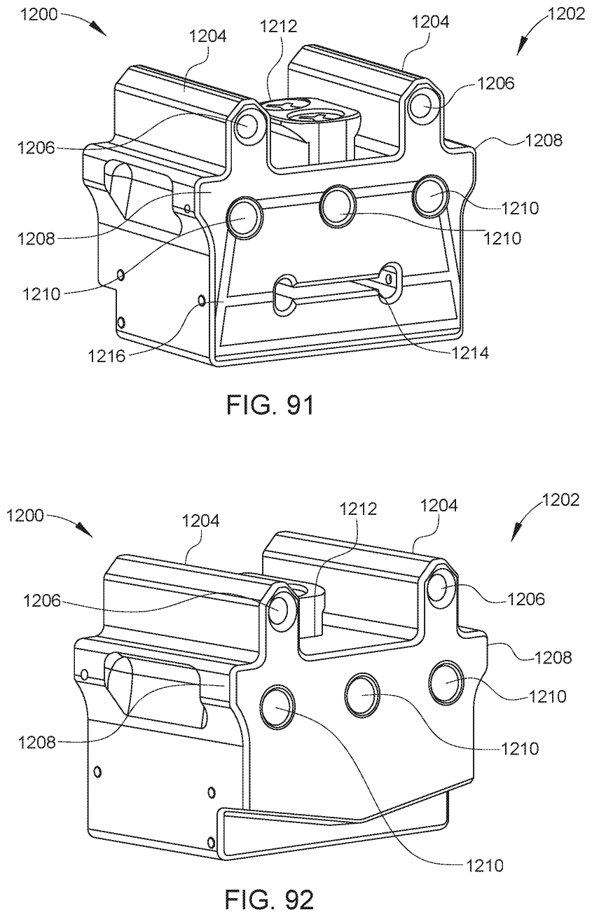

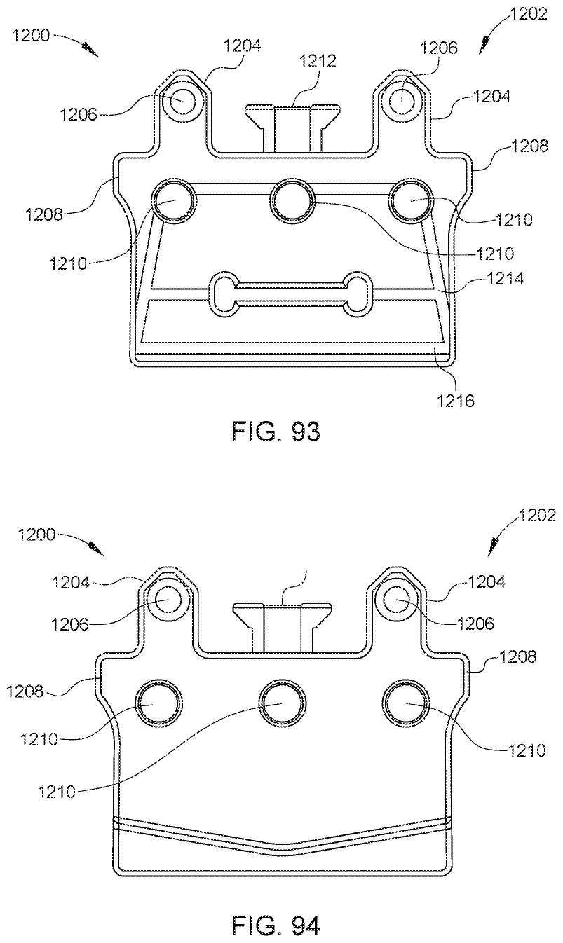

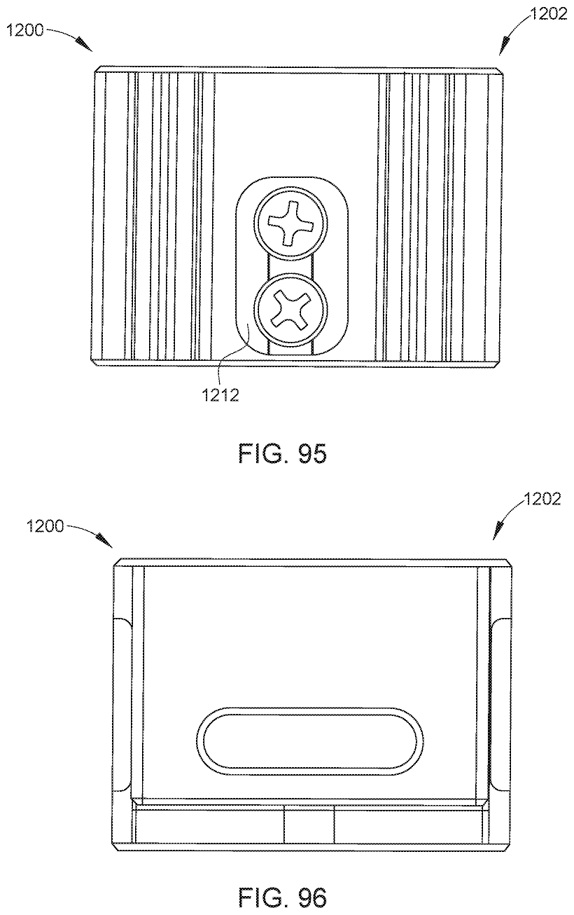

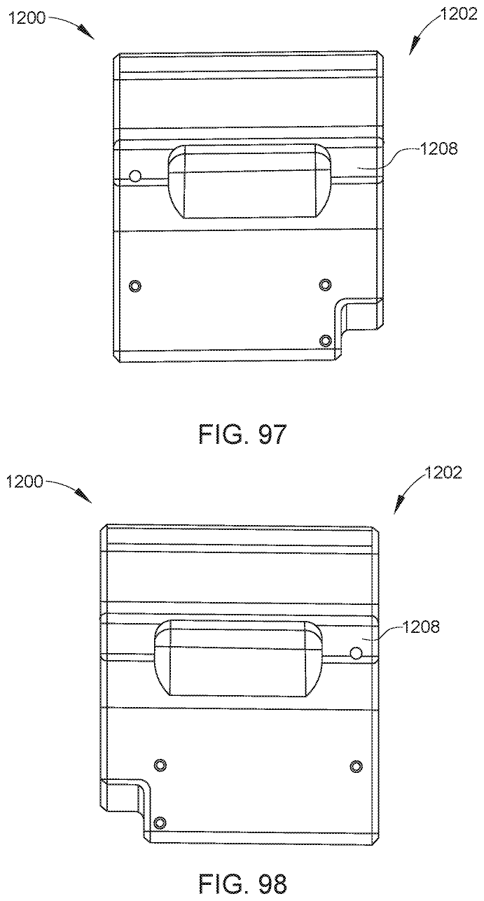

Referring now to FIGS. 90-98, an instrument 1200 is shown, according to an exemplary embodiment. The instrument 1200 may be couplable with one or more components of an ankle arthroplasty system such as alignment instruments, including but not limited to those incorporated by reference herein. In some aspects, the instrument 1200 may be releasably couplable with one or more such components, for example via one or more features of the instrument 1200. The instrument 1200 may be implemented in ankle arthroplasty procedure to guide at least one of drilling into the distal tibia and/or the talus of a patient, with the instrument 1200 configured to receive one or more cutting instruments at least partially therein and/or therethrough at least one opening/slot thereof. The instrument 1200 may also be configured to assist a physician in positioning and/or aligning the instrument 1200 and/or other components of an ankle arthroplasty system.

The instrument 1200 is shown to include a body 1202, from which a pair of extension 1204 extend upward in a direction substantially parallel to one another. Each of the extensions 1204 includes at least one opening 1206 extending therethrough from a front surface to a rear surface of the body 1202. The body 1202 further includes a pair of protrusions 1208 extending laterally from the body 1202 in directions substantially opposite one another. Each of the protrusions 1208 includes an opening 1210 positioned adjacent thereto and extending through from a top surface to a bottom surface of the body 1202. In some aspects, a third opening 1210 may be positioned centrally relative to the body 1202, equidistant and in a common plane to the aforementioned pair of openings 1210. The openings 1206 may include central axes which are substantially parallel to one another, whereas the openings 1210 may include central axes which converge (moving from a top/front surface of the instrument 1200 to a bottom/rear surface of the instrument 1200).

The instrument 1200 is further shown to include an engagement feature 1212 extending upward from the body 1202, according to one aspect. The engagement feature 1212, as shown in FIG. 83, is positioned substantially equidistant from and in between the pair of extensions 1204 and includes a male dovetail feature at a terminal portion thereof. In some aspects, the engagement feature 1212 may be configured to be received by a complementary feature of an alignment system so as to facilitate releasable coupling therewith (e.g., via a female dovetail component).

The body 1202 of the instrument 1200 includes a slot 1214 positioned centrally thereon and extending through the body 1202 from the top surface through to the bottom surface. As shown, the slot 1214 is substantially horizontal but in some aspects may be curved and/or angled. In some aspects, the slot 1214 may be substantially “dog bone” shaped, with two larger end portions separated by a linear portion. The slot 1214 may also be configured to receive one or more components therein, for example an angel wing or other releasably couplable alignment components. The body 1202 is further shown to include an outline 1016 disposed on a top surface of the body 1202 and intended to guide placement of the instrument 1200. In some aspects, the outline 1216 may include radiopaque ink (so as to be visible under x-ray/fluoroscopy), or may be indicative of the position of radiopaque elements within the body 1202 which facilitate positioning of the instrument 1200 under x-ray/fluoroscopy.

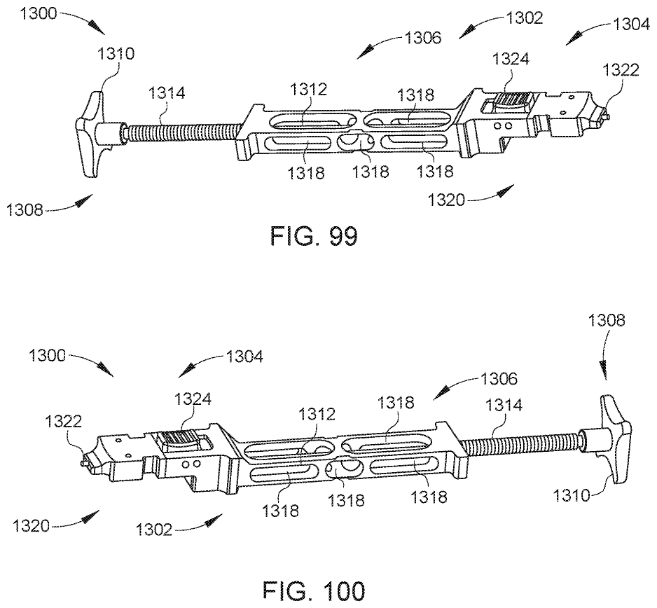

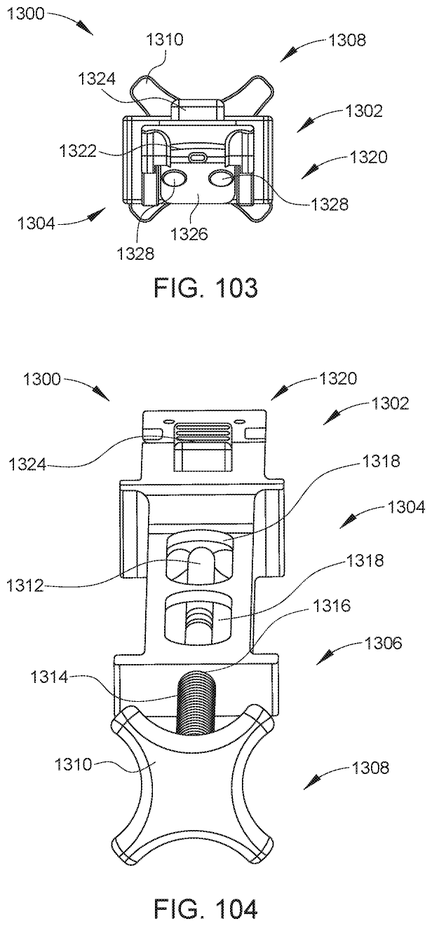

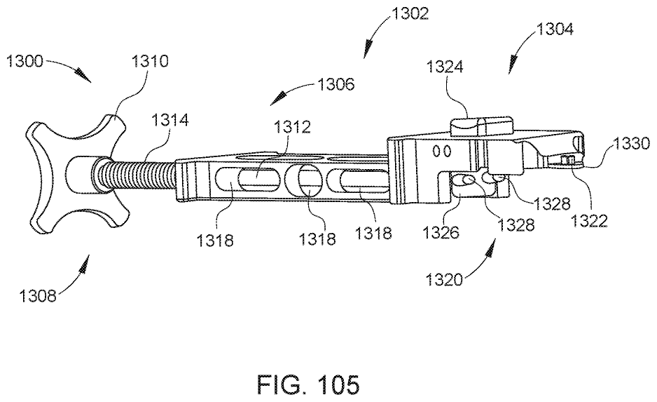

Referring now to FIGS. 99-105, an instrument 1300 (shown in an exemplary embodiment as an insertion instrument and referred to hereinafter as “inserter 1300) of a total ankle arthroplasty system is shown, according to an exemplary embodiment. In some aspects, the inserter 1300 may be provided as a component of a total ankle arthroplasty system, for example a total ankle arthroplasty system having one or more components with which the inserter 1300 is configured to engage. In some aspects, the inserter 1300 may include one or more features configured to engage with (e.g., releasably couple with) an intermediate implant component of a total ankle arthroplasty implant system. For example, the inserter 1300 may be configured to engage with a polymeric (e.g., “poly”) component to facilitate proper placement of the poly component relative to other components (e.g., tibial and talar implant components) of the total ankle arthroplasty implant system.

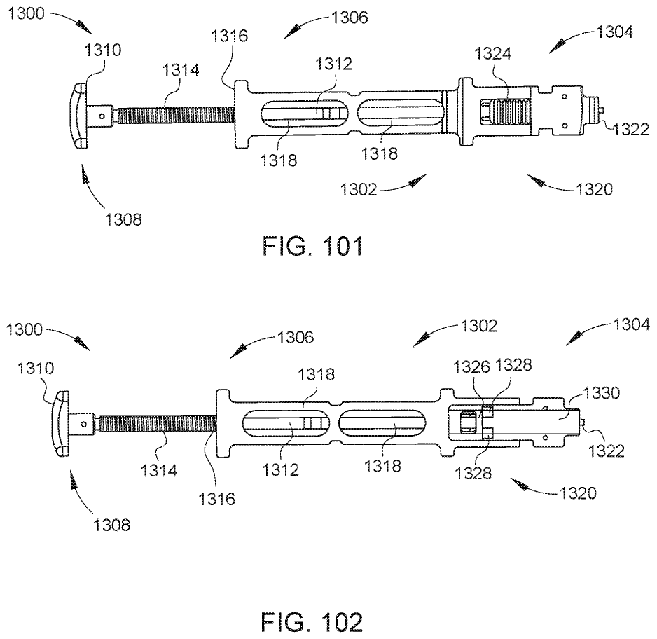

The inserter 1300 is shown to include a body 1302 which includes a distal portion 1304 and a proximal portion 1306, according to an exemplary embodiment. As shown in FIG. 100, at least a portion of the body 1302 has a substantially rectangular prism-shaped geometry. The distal and proximal portions 1304, 1306 are shown to be positioned at substantially opposite ends of the body 1302. In some aspects, the body 1302 may include a single, integral component where the distal and proximal portions 1304, 1306 are also integral with the body 1302 (as shown in at least FIG. 101). The body 1302 is shown to include openings 1318 positioned along the length of the body 1302 and on multiple sides of the body 1302. As shown, the openings 1308 are elongated openings extending from an outer surface of the body 1302 through to an inner cavity. In some aspects, the body 1302 may include multiple openings 1308 on one or more of the surfaces of the rectangular prism-shaped geometry thereof. For example, as shown in FIGS. 99-100, the top and bottom surfaces of the body 1302 include a pair of elongated openings 1308 positioned adjacent to one another, while each of the lateral surfaces includes three openings 1308 disposed thereon. As shown in at least FIG. 101, the openings 1308 of the top and bottom surfaces of the body 1302 have the same footprints so as to facilitate visibility through the entirety of the body 1302.

The inserter 1300 is shown to include an actuator 1308 extending from the proximal portion 1306 of the body 1302, where the actuator 1308 includes a handle portion 1310 and a shaft portion 1312. In some aspects, the handle portion 1310 may include one or more features to facilitate gripping and/or actuating (e.g., rotating) of the handle portion 1310 by a physician. For example, as shown in FIG. 100, the handle portion 1310 includes four protrusions separated from one another by curvatures to facilitate use thereof by a physician. The shaft portion 1312 is shown to include a threading 1314 positioned on a proximal portion of the shaft portion 1312 and configured to engage with a threaded opening 1316 disposed in the distal portion 1306 of the body 1302. Accordingly, actuation of the handle portion 1310 engages the threading 1314 with the threaded opening 1316, and thus translates the shaft portion 1312 of the actuator 1308 along an axis that is coaxial with that of the threaded opening 1316. Translation of the shaft portion 1312 within the body 1302 can be visualized through one or more of the openings 1308 of the body 1302. In some aspects, the shaft portion 1312 may include one or more laser marks disposed thereon so as to provide reference for the user as to the translation of the shaft portion 1312 as a result of the rotation of the handle portion 1310. For example, one or more laser marks may be configured to indicate partial or full seating of the poly component relative to the tibial component when aligned with (e.g., coplanar with) corresponding points or marks on the body 1302.

The distal portion 1304 of the body 1302 is shown to include an engagement portion 1320 positioned at a distal-most end thereof. The engagement portion 1320 is shown to include a projection 1322 extending in a proximal direction therefrom and is configured to be received at least partially within a component of a total ankle arthroplasty implant system (e.g., a poly component). The distal portion 1304 is also shown to include a locking mechanism 1324 which, when actuated, is configured to facilitate releasably coupling between the projection 1322 and the aforementioned component of a total ankle arthroplasty implant system. The locking mechanism 1324 may be actuated by pressing on the surface of the component positioned adjacent to the top surface of the engagement portion 1320, and may be translated in a forward/backward motion in directions substantially parallel to that of the axis of the shaft portion 1312.

The inserter 1300 is further shown to include a translating element 1326, which is shown to be engaged with an end of the shaft portion 1312 opposite the handle portion 1310. In some aspects, the translating element 1326 may be integral with the shaft portion 1312, or the translating element 1326 may be coupled with the distal-most end of the shaft portion 1312. As shown, the translating element 1326 includes a pair of projections 1328 extending from a surface thereof in a direction substantially opposite that of the shaft portion 1312. As shown, each projection of the pair of projections 1328 is substantially the same, but in some aspects one projection of the pair of projections 1328 may be different from the other projection. Accordingly, upon actuation of the handle portion 1310, the shaft portion 1312 is threadably translated toward the distal-most end of the inserter 1300 via the threading 1314 and the threaded opening 1316 such that the translating element 1326 is moved along a bottom surface 1330 of the engagement portion 1320 toward the projection 1322.

In implementing the inserter 1300, a physician may actuate the handle portion 1010 (e.g., in a counterclockwise direction) to withdraw the shaft portion 1012 (and accordingly, the translating element 1326) to its proximal-most position (e.g., with as much of the threading 1314 positioned between the handle portion 1310 and the threaded opening 1316 as possible). The physician may then manipulate the inserter 1300 such that the projection 1322 is received at least partially within a complimentary opening of a component of a total ankle arthroplasty implant system, for example an intermediate poly component. The physician may then engage the locking mechanism 1324 so as to retain the projection 1322 at least partially within the opening of the poly component. The physician may then manipulate the inserter 1300 so as to position the poly component adjacent to a desired location for the poly component, for example just below a tibial implant component that has already been implanted in the distal tibia of a patient. In some aspects, the poly component may releasably couple or otherwise engage with the tibial implant and, accordingly, features of one or both components that accommodate the engagement/coupling (e.g., a dovetail, etc.) may be aligned adjacent to one another. The physician may then actuate the handle portion 1310 in a clockwise direction, thus translating the shaft portion 1312 and the translating element 1326 toward the poly component with which the projection 1322 of the inserter 1300 is engaged. Accordingly, one or both of the projections 1328 of the translating element 1326 may contact a front surface of the poly component. At this time, the physician may manipulate the locking mechanism 1324 such that the poly component may be decoupled from the projection 1322 as at least a portion of the poly component is engaged with the tibial implant component via a dovetail or other means of releasable coupling. Accordingly, the handle portion 1012 may be rotated in a clockwise direction again such that the projections 1328 of the translating element 1326 bias the poly component into a desired position that is both engaged with and positioned below the tibial implant component. The inserter 1300 may then be manipulated such that the projection 1322 is withdrawn from the corresponding opening in the poly component.

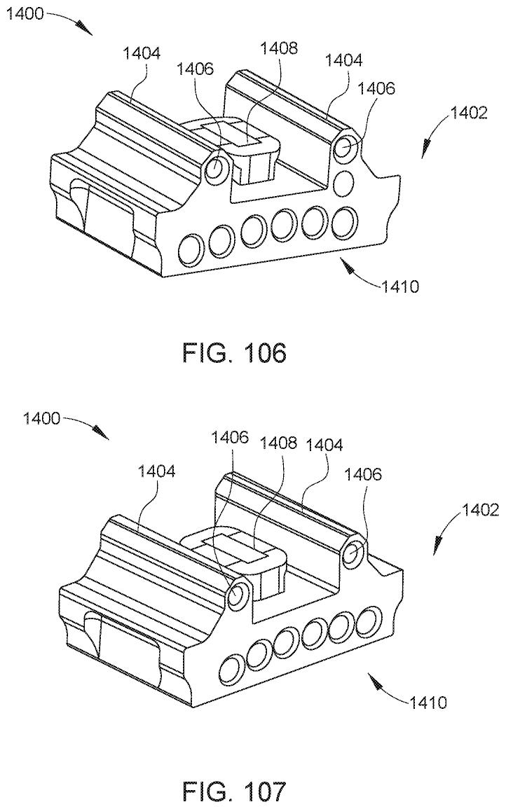

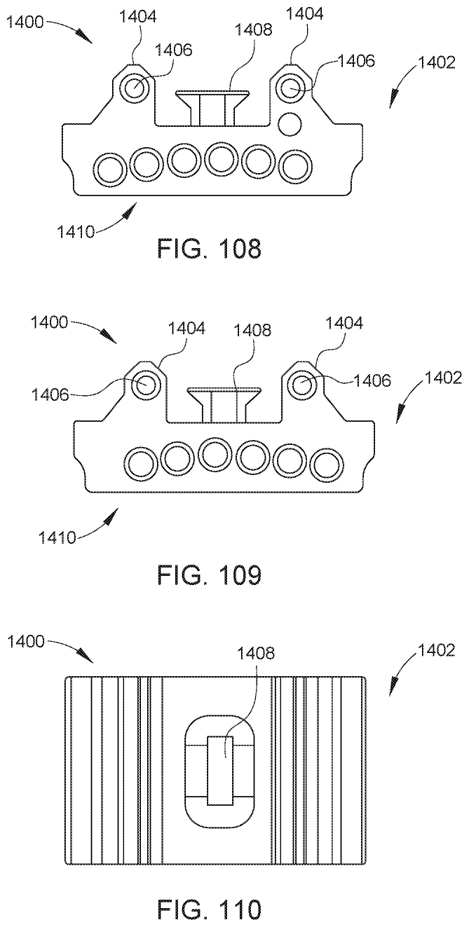

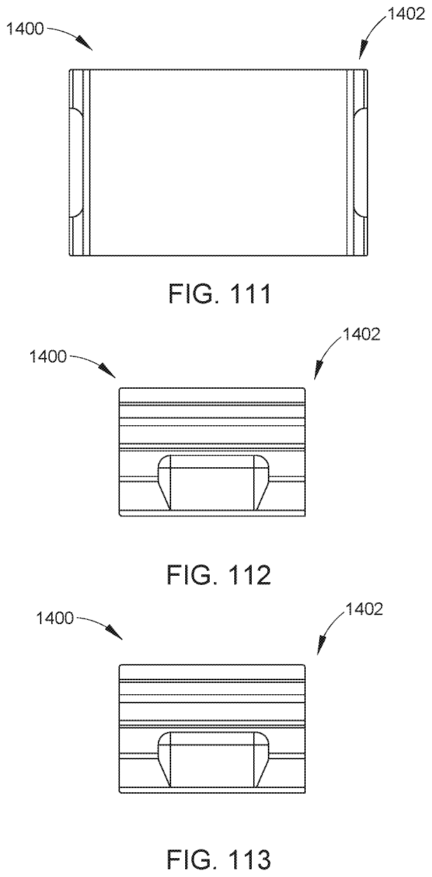

Referring now to FIGS. 106-113, an instrument 1400 is shown, according to an exemplary embodiment. The instrument 1400, which is shown and described herein as a drill guide, may be compatible (e.g., releasably couplable) with one or more other elements of a total ankle arthroplasty system, for example an alignment guide or other similar instrumentation. In some aspects, the instrument 1400 may have cross-compatibility with various instrumentation of the total ankle arthroplasty system including, for example, one or more drill bits and/or stabilization wires (e.g., k-wires, olive wires, etc.).

The instrument 1400 includes a body 1402, which is integral with a pair of projections 1404 extending upward from a top surface of the body 1402. As shown, the projections 1404 extend along axes parallel to one another, and are separated by an engagement feature 1408 which, as shown in the exemplary embodiment herein, is a dovetail feature. In some aspects, the engagement feature 1408 may include alternate engagement mechanisms, for example various geometric features of which an alignment instrument of the total ankle arthroplasty system may include complementary geometric features to facilitate releasable coupling therebetween. As shown, each of the projections 1404 includes an opening 1406 extending therethrough from a top surface to a bottom surface of the projection 1404. The central axes of the openings 1406 are substantially parallel (e.g., to facilitate sliding over a set of already-placed pins), but in some aspects the axes may be substantially converging or diverging.

The body is further shown to include openings 1410 extending from a front surface of the body 1402 through to a rear surface of the body 1402. As shown, the body 1402 includes six openings 1410, but in some aspects the body 1402 may include alternate numbers of openings 1410. Similarly, the openings 1410 are shown to be positioned with equidistant spacing from one another along a substantially arced trajectory. However, in some aspects the openings 1410 may be positioned variously about the body 1402. Each of the openings 1410 are shown to receive a drill bit at least partially therethrough, so as to guide or otherwise facilitate drilling into the anatomy positioned adjacent to the instrument 1400 (for example, the distal tibia). Further, the instrument 1400 is configured such that it is reversible. For example, the instrument 1400 may be coupled with a distal tibia of a patient and/or an alignment system for a total ankle arthroplasty system. Holes may then be drilled in the distal tibia through the openings 1410. The instrument 1400 may then be decoupled, flipped, and recoupled as it was coupled previously. Holes may then be drilled into the distal tibia through the holes 1410 again. It should be noted that the second set of holes drilled in the distal tibial will be offset from the first set of holes drilled in the distal tibia, as the openings 1410 are slightly biased toward one lateral edge of the body 1402 (and thus are not centered laterally on the body 1402).

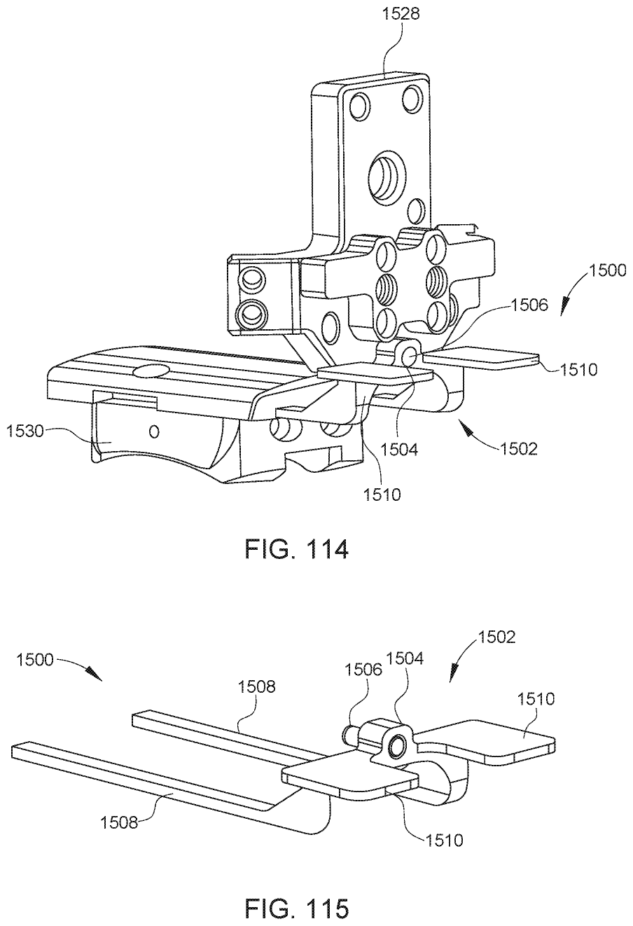

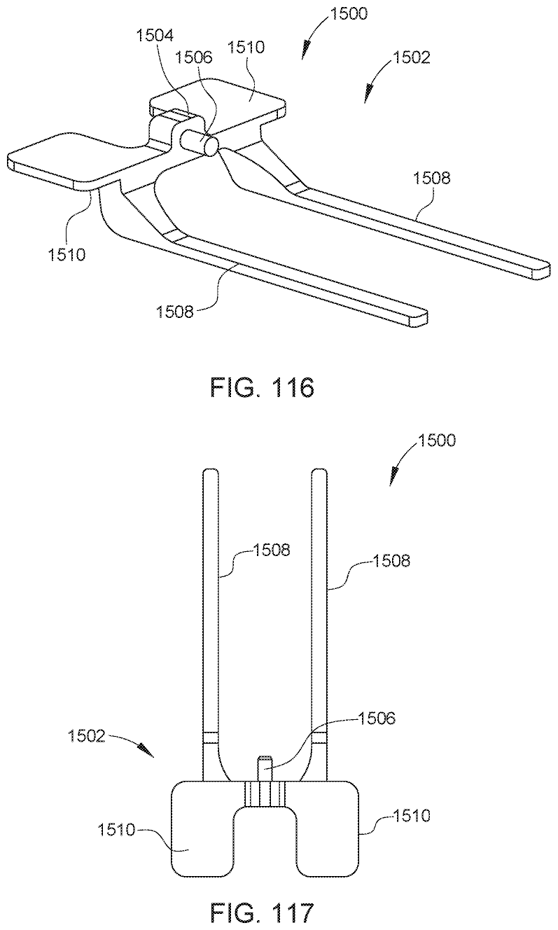

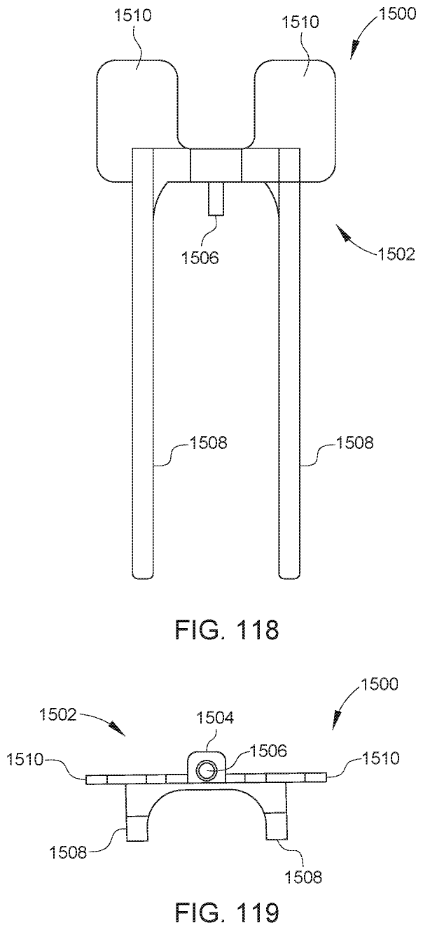

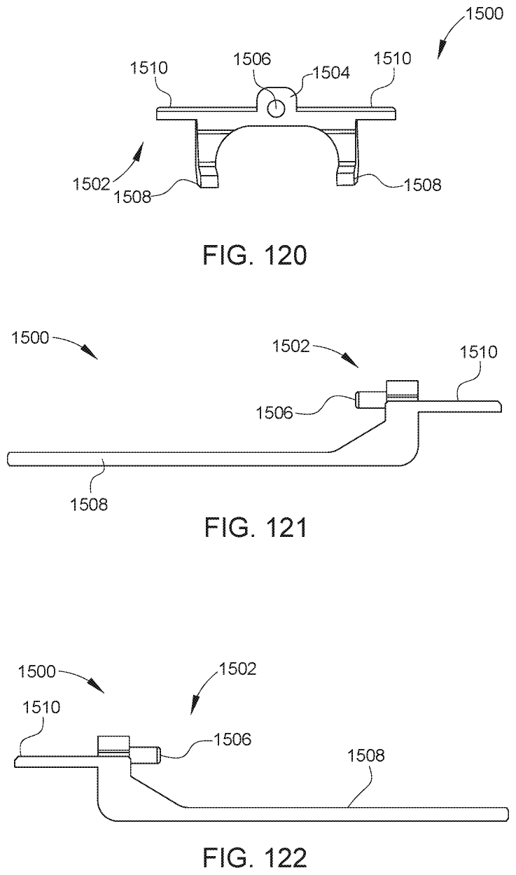

Referring now to FIGS. 114-122, an instrument 1500 is shown, according to an exemplary embodiment. The instrument 1500, as shown in FIG. 114, is configured to releasably couple/engage with various instrumentation of a total ankle arthroplasty system including, for example, a tibial alignment guide 1528 and a trial insert 1530. The instrument 1500 is shown to include a body portion 1502, which includes an opening 1504 extending through a central portion thereof from a front surface through to a rear surface. As shown in at least FIGS. 114-115, the opening 1504 is configured to receive a coupling element 1506 at least partially therethrough so as to facilitate releasable coupling with at least the tibial alignment guide 1528. As shown, the coupling element has a substantially cylindrical geometry, but in some aspects may have alternate geometries.

The body 1502 is shown to include a pair of legs 1508 extending below and away from the rear surface of the body 1502. As shown, the pair of legs 1508 are substantially parallel to one another, but in some aspects may be converging or diverging. Further, the body 1502 is shown to include a pair of wings 1510 extending laterally from opposite sides of the body 1502. As shown, each of the wings 1510 has a substantially rectangular geometry, although in some aspects the wings 1510 may have alternate geometries. The top surface of the wings 1510 is shown to be positioned in a plane that is substantially parallel to that of the top surface of the legs 1508.

The terminology used herein for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

As may be recognized by those of ordinary skill in the art based on the teachings herein, numerous changes and modifications may be made to the above-described and other embodiments of the present disclosure without departing from the scope of the disclosure. The components of the implants as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants may include more or fewer components or features than the embodiments as described and illustrated herein.

What is claimed is:

1. An ankle arthroplasty implant, comprising:
a body, comprising:
a top surface opposite a bottom surface;
a lateral surface opposite a medial surface; and
a front end opposite a rear end; and
an extension extending upward from a central portion of the top surface of the body.

2. The implant of claim 1, wherein the extension is integral with the body of the implant.

3. The implant of claim 2, wherein the extension further comprises:
a base positioned at a lower portion of the extension;
a top portion positioned at an upper portion of the extension; and
a shaft positioned between the base and the top portion.

4. The implant of claim 3, wherein the top portion of the extension is integral with the shaft of the extension.

5. The implant of claim 3, wherein the top portion comprises a rounded geometry along at least a portion thereof.

6. The implant of claim 5, wherein the shaft comprises a circular cross-sectional geometry along at least a portion of the length of the shaft.

7. The implant of claim 6, wherein the shaft is integral with the body of the implant.

8. The implant of claim 6, wherein the base extends upward from the top surface of the body such that the base extends around at least a portion of an outer surface of the shaft.

9. The implant of claim 8, wherein the base extends upward from the top surface of the body such that the base extends around the entirety of the outer surface of the shaft along at least a portion of the length of the shaft.

10. The implant of claim 8, wherein the base comprises a cannulation through which at least a portion of the shaft extends.

11. The implant of claim 10, wherein the top surface of the base forms a substantially oblique angle with the outer surface of the base and with the outer surface of the shaft.

12. The implant of claim 11, wherein the base comprises an at least partially porous lattice geometry through at least a portion of the base.

13. The implant of claim 12, wherein the base is integral with the body of the implant.

14. The implant of claim 11, wherein the body comprises an at least partially porous lattice geometry through at least a portion of the body.

15. The implant of claim 14, wherein the base and body comprise the same at least partially porous lattice structure, which extends through at least a portion of the body and at least a portion of the base.

16. The implant of claim 15, wherein the top surface of the body comprises a substantially flat surface.

17. The implant of claim 15, wherein the top surface of the body comprises a substantially arced surface.

18. The implant of claim 17, wherein the body of the implant comprises a radiused portion between the top surface and each of the medial and lateral surfaces.

19. An ankle arthroplasty implant, comprising:
a body comprising an at least partially porous lattice structure extending through at least a portion of the body, the body further comprising:
a top surface opposite a bottom surface;
a lateral surface opposite a medial surface; and
a front end opposite a rear end; and
an extension extending upward from a central portion of the top surface of the body, wherein the extension comprises:
a shaft portion integral with the body and comprising a substantially cylindrical geometry;
a top portion positioned at and integral with a terminal end of the shaft portion, wherein the top portion comprises a rounded geometry along at least a portion of an outer surface thereof; and
a base portion extending upward from and integral with the body of the implant, wherein the base portion extends around at least a portion of an outer surface of the shaft portion, wherein the base portion comprises an at least partially porous lattice structure through at least a portion of the base portion.

20. The implant of claim 19, further comprising a dovetail feature disposed on the bottom surface of the body of the implant.

21. An ankle arthroplasty kit, comprising:
a plurality of tibial implants;
a plurality of poly bearing inserts for insertion into the plurality of tibial implants;
a tibial alignment guide for coupling to at least one of the plurality of tibial implants;
a drill guide; and
a poly bearing insert inserter for use with at least one of the tibial implants.
